(19) United States
(12) Patent Application Publication
(10) Pub. No.: US 2014/0051678 A1
(43) Pub. Date: Feb. 20, 2014

(54) INHIBITORS OF THE ACTIVITY OF COMPLEX III OF THE MITOCHONDRIAL ELECTRON TRANSPORT CHAIN AND USE THEREOF FOR TREATING DISEASES

(71) Applicants: Virginie Clement-Schatlo, Suhr (CH); Thomas Fessard, Zürich (CH); Riccardo Cribiu, Zürich (CH)

(72) Inventors: Virginie Clement-Schatlo, Suhr (CH); Thomas Fessard, Zürich (CH); Riccardo Cribiu, Zürich (CH)

(73) Assignee: STEMERGIE BIOTECHNOLOGY SA, Geneva (CH)

(21) Appl. No.: 13/989,319

(22) PCT Filed: Nov. 24, 2011
(86) PCT No.: PCT/IB11/55287

§ 371 Date: Aug. 6, 2013

(30) Foreign Application Priority Data
Nov. 24, 2010 (CH) 01965/10

(51) Int. Cl. C07D 491/107; A61K 31/365; A61K 31/397; A61K 31/167; C07D 267/00; C07D 321/00; C07C 235/62

(52) U.S. Cl. CPC C07D 491/107; C07D 321/00; A61K 31/365; C07C 235/62; A61K 31/167; C07D 267/00; A61K 31/397
USPC 514/210.19; 549/267; 514/450; 564/158; 514/616; 540/455; 548/950

(57) ABSTRACT

The present invention relates to compounds which are inhibitors of the activity of Complex III of the mitochondrial electron transport chain and pharmaceutical compositions comprising said compounds alone or in combination with other active agents. The present invention further relates to use of the compounds of the invention as medicaments or as agrochemicals where their properties as inhibitors of the mitochondrial respiration is of benefit. More particularly the present invention relates to the use of the compounds of the invention in a method of treating and/or preventing cancers presenting tumor-initiating cells. (Formula I) (I)

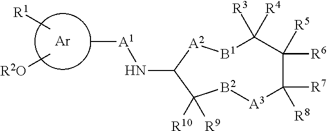

### FIELD OF THE INVENTION

The present invention relates to compounds which are inhibitors of the activity of Complex III of the mitochondrial electron transport chain and pharmaceutical compositions comprising said compounds alone or in combination with other active agents. The present invention further relates to use of the compounds of the invention as medicaments or as agrochemicals where their properties as inhibitors of the mitochondrial respiration is of benefit. More particularly the present invention relates to the use of the compounds of the invention in a method of treating and/or preventing cancers presenting tumor-initiating cells.

### BACKGROUND OF THE INVENTION

Cancer affects people at all ages with the risk for most types increasing with age. Cancers are mainly due to genetic deregulations of cells and to lifestyle and environmental factors., which cause abnormalities in the genetic material of cells.

For example brain tumors make up to 2% of all tumors in adults and, in their malignant form (grade IV or glioblastoma (GBM)) remain one of the most aggressive diseases with a 2-years survival rate of 32% with today's available standard treatments. It is reported that 1 out of 166 humans are diagnosed with brain tumor once in their lifetime (lifetime risk). Although combining chemotherapy with radiation shows a significant benefit for patients suffering from glioblastoma (GBM), the mean survival rate regains dismal, 16 months on average. Neither genetic factors nor environmental risk factors have been identified and little is known about the biological mechanisms involved in the initiation and progression phases of these brain tumors.

The treatment of cancers is one of the most heavily investigated areas in biomedical research today. Although many anticancer drugs have been and continue to be discovered, there remains the immense problem of developing drugs which will efficiently address this disease and avoid it recurrence.

Many current therapeutic strategies make the assumption that the biology and metabolism of every single cancer cell, including glioma cells, is similar and unfortunately did not provide a significant progress in the treatment of cancers, including glioma.

The recent identification of Stera-like Cells (SC) in a number of human cancers like acute myeloid leukemias (AML), breast, melanoma, colon and brain tumors has renewed interest in the hypothesis that cancers may arise from somatic mutations in adult stem/progenitor cells. A minor population of cancer stem-like cells is likely to represent the source of tumor cell expansion, recurrence and metastasis, thus determining the biological behaviour of tumors including proliferation, progression, and subsequently response to therapy.

Brain tumor cancer initiating cells, know as Glioma-initiating cells (GICs) were initially identified as CD133⁺ cells but recent studies demonstrate a relative lack of specificity of this marker. These cells are heterogeneous populations of cells with different tumorigenic capacity, some tumor cells having a superior tumor initiating and propagating ability. Glioma-initiating cells (GICs) are responsible for the initiation and recurrence of gliomas. The role of glioma-initiating cells with stem cell properties has not yet been well investigated. These cells display characteristic stem cell features including self renewal capacity at single cell level, multipotency with evidence of astroglial, neuronal and oligodendroglial differentiation in vitro and tumorigenicity in vivo. As other human cancers, gliomas contain cellular hierarchies on the top of which tumor initiating and propagating cells with stem cell properties (called cancer stem cells-CSC) seem to control tumor growth. This minor population of cancer stem-like cells, GICs account only for about 5% of tumor cells (gliomas), may represent the source of tumor cell expansion, recurrence and metastasis, thus determining the biological behaviour of tumors including proliferation, progression, and subsequently response to therapy.

Targeting tumor-initiating cells remains challenging due to their rarity, instability in culture and the absence of robust tracer agents. Namely, glioma-initiating cells present a very-minor cell population which is not possible to easily target in the glioma (tumor) bulk. So far, no efficient treatment against tumor-initiating cells has shown a complete eradication of the tumor growth or absence of recurrence in any of the orthotopic xenograft and/or transgenic mouse model, The resistance of tumor-initiating cells to conventional radiotherapy has been demonstrated (Bao et al., 2006; Clement et. al., 2007), For example it is known that glioma-initiating cells are resistant to chemotherapeutic agents like temozolomide. These data might explain the inevitable recurrence of gliomas and define tumor-initiating cells as novel targets to overcome the resistance to conventional therapy in this disease.

US 2007/0123448 discloses neuroblastoma tumor-initiating cell inhibiting compositions comprising chemical entities capable of affecting neuroblastoma tumor-initiating cells. Neuroblastoma is the most common extracranial solid tumor in childhood and the most common cancer in infancy. In contrast to glioma, it is a neuroendocrine tumor, arising from any neural crest cells of the sympathetic nervous system. However it does not seem that the disclosed compounds were tested and therefore show an efficient targeting of neuroblastoma tumor-initiating cells.

EP 138834 2 A1 discloses acylated, heteroaryl-condensed cycloalkenylamines of the formula I,

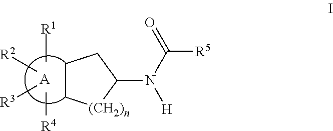

These compounds are disclosed as valuable pharmacologically active compounds which are useful in the treatment of various disease states including cardiovascular disorders such as atherosclerosis, thrombosis, coronary artery disease, hypertension and cardiac insufficiency. They upregulate the expression of the enzyme endothelial nitric oxide (NO) synthase and can be applied in conditions in which an increased expression of said enzyme or an increased NO level or the normalization of a decreased NO level is desired. However these compounds are not shown to target tumor-initiating cells.

EP 1054011 A1 discloses compounds having antimicrobial activity of formula

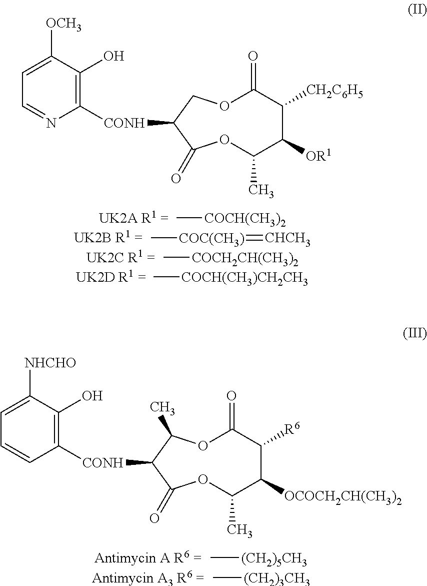

However EP 1054011 A1 does not disclose and does not show the use of these compounds to target tumor-initiating cells and to treat and/or prevent cancers presenting tumor-initiating cells.

Because it was shown that targeting tumor-initiating cells (TICs) is not easy and that these cells are actively chemo- and radio-resistant, including brain tumors, there is therefore a need to develop new active agents against the TICs reservoir which may be useful in the treatment of diseases, such cancers presenting tumor-initiating cells.

### SUMMARY OF THE INVENTION

The present invention relates to a compound of formula I

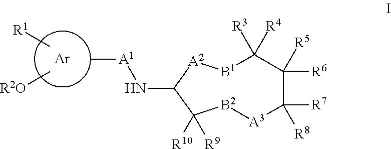

or a pharmaceutically acceptable salt or solvate thereof, wherein

Ar is selected from (C5-C10) aromatic ring or (C5-C10) heteroaromatic ring where one or more of the carbon atoms in the ring system are replaced by heteroatoms selected from the group consisting of O, S, and N;

R¹ is selected from H, —(C1-C10)alkyl, -aryl, —OH, —O—(C1-10)alkyl, —O-aryl, —NH₂, —NH(CHO), —NH(C═O)—(C1-10)alkyl, halogen, —NO₂, —C(═O)OH, —C(═O)O(C1-10)alkyl, —CF₃, —NH(C═O)(CF₃(OMe))Ph;

R³ is selected from —H, —(C1-10)alkyl, aryl, benzyl, —(C═O) (CF₃ (OMe))Ph;

R³ is selected from —H, —(C1-10)alkyl, —CF₃ or can form a bond with R⁵;

R⁴, R⁹ and R¹⁰ are independently of each other selected from —H, —(C1-10)alkyl, —CF₃,

A¹, A² and A³ are independently of each other selected from —(C═O)—, —(C═S)—, —(S═O)—, —(S(═O)₂)—, —(CH₂)—, —(C(CH₃)₂)—, —CH(CF₃)—, —CHF—, —(C(CH₂OCH₂))—, —(C(CH₂SCH₂))—, —N(R^{a})—, —(CR′R″)—, —CH₂—CH₂—, —(C(CH₃)₂)—, where R^{a} is selected from —H, —(C1-C10)alkyl, —(C1-C10)alkenyl, —(C1-C10)alkynyl, mono or polyfluorinated (C1-C10)alkyl, —(C3-C10)cycloalkyl, —(C3-C10)heterocycloalkyl, -aryl, —(C1-C10)alkylaryl, —(C1-C10)alkylheteroaryl, —(C1-C10)alkyl-heterocycloalkyl, and where R′ and R″ are independently of each other selected from —H, —(C1-C10) alkyl, -halogen, —CF₃;

B¹ and B²:
(i) are independently of each other selected from —O—, —S—, —NH—, —CH₂—, or
(ii) form together —CH—CH— producing a bicyclo(3,4,0)-nonane unit
(iii) form together —C═C— producing a bicycle
(iv) form together a —N—N— or —CH—N— fragment producing a diazabycycle or azabicycle

R⁵, R⁶, R⁷ and R⁸ are selected from:
(i) independently of each other, —R^{a}, —OR^{a}, —O(C═O)R^{a}, —C(═O)R^{a}, —C(═O)OR^{a}, —NR^{a}R^{b}, —NR^{a}(C═O)R^{b}, halogen, where R^{a} and R^{b} are independently of each other selected from —H, —(C1-C10)alkyl, —(C1-C10) alkenyl, —(C1-C10)alkynyl, mono or polyfluorinated (C1-C10)alkyl, —(C3-C10)cycloalkyl, —(C3-C10)heterocycloalkyl, -aryl, —(C1-C10)alkylaryl, —(C1-C10)alkylheteroaryl, —(C1-C10)alkyl-heterocycloalkyl or a combination thereof, or;
(ii) R⁵ and R⁷ form together a bond AND R⁶ and R⁸ are independently of each other selected from —R^{a}, —OR^{a}, —O(C═O)R^{a}, —C(═O)R^{a}, —C(═O)OR^{a}, —NR^{a}R^{b}, —NR^{a}(C═O)R^{b}, halogen, where R^{a} and R^{b} are independently of each other selected from —H, —(C1-C10)alkyl, —(C1-C10)alkenyl, —(C1-C10)alkynyl, mono or polyfluorinated (C1-C10)alkyl, —(C3-C10)cycloalkyl, —(C3-C10)heterocycloalkyl, -aryl, —(C1-C10)alkylaryl, —(C1-C10)alkylheteroaryl, —(C1-C10)alkyl-heterocycloalkyl or a combination thereof, or;
(iii) R⁵ and R⁷ represent independently of each other, —R^{a}, —OR^{a}, —O(C═O)R^{a}, —C(═O)R^{a}, —C(═O)OR^{a}, —NR^{a}R^{b}, —NR^{a}(C═O)R^{b}, halogen, where R^{a} and R^{b} represent independently of each other —H, —(C1-C10)alkyl, —(C1-C10)alkenyl, —(C1-C10)alkynyl, mono or polyfluorinated (C1-C10)alkyl, -aryl AND R⁶ and R⁸ form together a (C3-C10)cycloalkyl or (C3-C10)heterocycloalkyl substituted by R^{c} and R^{d} where R^{c} and R^{d} represent independently of each other —H, —(C1-C10)alkyl, —(C1-C10)alkenyl, —(C1-C10)alkynyl, mono or polyfluorinated (C1-C10)alkyl, -aryl, —OH, —O—(C1-C10)alkyl, —O-aryl, —O(C═O)—(C1-C10)alkyl, —O(C═O)-aryl, halogen, or;
(iv) R⁵ and R⁷ form together a bond AND R⁶ and R⁸ form together a (C3-C10)cycloalkyl or heterocycloalkyl substituted by R^{c} and R^{d} where R^{c} and R^{d} represent independently of each other —H, —(C1-C10)alkyl, —(C1-C10)alkenyl, —(C1-C10)alkynyl, mono or polyfluorinated (C1-C10)alkyl, -aryl, —OH, —O—(C1-C10) alkyl, —O-aryl, —O(C═O)—(C1-C10)alkyl, —O(CO)-aryl, halogen, or;
(v) R⁵ and R⁷ form together a bond AND R⁶ and R⁸ form together an (C6-C10)aryl or (C5-C10)heteroaryl substituted, by R^{c} and R^{d} where R^{c} and R^{d} represent independently of each other —H, —(C1-C10)alkyl, —(C1-C10)alkenyl, —(C1-C10)alkynyl, mono or polyfluorinated (C1-C10)alkyl, -aryl, —OH, —O—(C1-C10)alkyl, —O-aryl, —O(C═O)—(C1-C10)alkyl, —O(C—O)-aryl, halogen, or;
(vi) when B¹ and B² form together —C═C—, R³ and R⁵ form together a bond AND A³ is —C(R′R″)— or —N(R′)— AND R⁷ and R′ form together a bond, where R′ and R″ are independently of each other selected from —H, —(C1-C10)alkyl, -halogen, —CF₃;
   with the provisos that
(i) when A¹, A² and A³ are —C(═O)— AND B¹ and B² are —O— AND R³, R⁵, R⁷ and R⁹ are —H, the compound of formula I excludes the compound of formula II

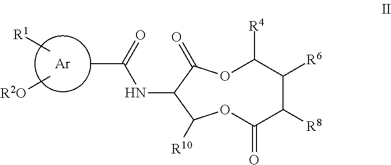

wherein
   Ar is an aromatic ring selected from benzene, pyridine, indazole, benzotriazole, benzymidazole or pyrazolothiophene;
   R¹ is selected from H, —C(1-10)alkyl, —O(C1-C10)alkyl, —Cl, —NO₂, —NH₂, —NHC(═O)H, —NH(C═O)—C(1-10) alkyl, —N[(C1-C10)alkyl]₂, —N[C(1-10)alkyl]C(═O)C(1-10)alkyl, —NH(C═O) (CF₃(OMe))Ph;
   R² is selected from H, —C(1-10) alkyl, phenacyl, benzyl, benzoyl, —(C═O) (CF₃(OMe))Ph;
   R⁴ and R¹⁰ are independently of each other selected from —H or —(C1-C10)alkyl;
   R⁶ and R⁸ are independently of each other selected from —H, —(C1-C10)alkyl, —OH, —O(C1-C10)alkyl, —OC(═O)(C1-C10)alkyl, —O-benzyl, —OC(═O)aryl, —OCO(CH₃)═CHCH₃, —CH₂C₆H₅, or —O(C═O) benzyl wherein —(C1-C10)alkyl, —O(C1-C10)alkyl, —OC(═O)(C1-C10)alkyl, —O-benzyl, —OC(═O)aryl and —O(C═O) benzyl are optionally substituted with —OH, -halogen, —NO₂, —CO₂H;
(ii) when A¹, A² and A³ are —C(═O)— AND B¹ and B² are —O— AND R³ and R⁹ are —H AND R⁵ and R⁷ form together a bond AND R⁶ and R⁸ form together an aryl ring, the compound of formula I excludes the compound of formula III

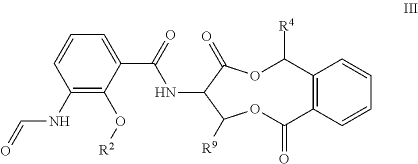

wherein
   R², R⁴ and R⁹ are independently of each other selected from —H or —(C1-C10)akyl;
(iii) when A¹ is —C(═O)— AND A² is —CH₂— AND A³ is —C(R′R″)— AND R⁹ and R¹⁰ are —H AND B¹ and B² form together a —C═C— AND R³ and R⁵ and R⁷ and R′ form together a bond, the compound of formula I excludes the compound of formula IV

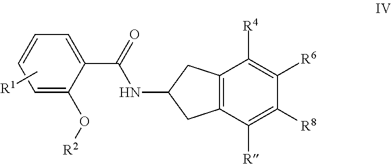

wherein
   R″, R⁴, R⁶ and R⁸ are independently of each other selected from —H or —(SO₂)—R^{e} where R^{e} is selected from -halogen, —(C1-C10)alkyl, -cycloalkyl, heterocycloalkyl, -aryl, -heteroaryl, —NH₂, —OR or combination thereof;
   R¹ is selected from H, —(C1-C10)alkyl, -aryl, —OH, —O—(C1-10)alkyl, —O-aryl, —NH₂, —NH(CHO), —NH(C═O)—(C1-10)alkyl, halogen, —NO₂, —C(═O)OH, —C(═O)O(C1-10)alkyl, —CF₃, —NH(C═O) (CF₃(OMe))Ph;
   R² is selected from H, —(C1-10)alkyl, aryl, benzyl, —(C═O) (CF₃(OMe))Ph;
(iv) when A¹ is —C(═O)— AND A³ is —H(R′)— AND R⁹ and R¹⁰ are —H AND B¹ and B² form together a —CH—N— fragment AND R³, R⁶ and R⁷ are —H, the compound of formula I excludes the compound of formula V

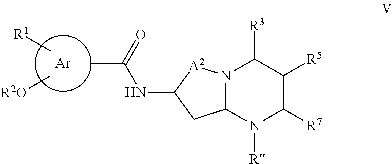

wherein
   A² is selected from —CH₂— or —C(═O)—
   Ar is selected from (C5-C10) aromatic ring or (C5-C10)heteroaromatic ring;
   R¹ is selected from H, —(C1-C10)alkyl, -aryl, —OH, —O—(C1-10)alkyl, —O-aryl, —NH₂, —NH(CHO), —NH(C═O)—(C1-10)alkyl, halogen, —NO₂, —C(═O)OH, —C(═O)O(C1-10)alkyl, —CF₃, —NH(C═O) (CF₃ (OMe))Ph;
   R₂ is selected from H, —(C1-10)alkyl, aryl, benzyl, —(C═O)(CF₃(OMe))Ph;
   R³ is selected from —H, —(C1-10)alkyl, —CF₃ or can form a bond with R⁵;
   R⁵ and R⁷ are selected from independently of each other, —R^{a}, —OR^{a}, —O(C═O)R^{a}, —C(═O)R^{a}, —C(═O)OR^{a}, —NR^{a}R^{b}, —NR^{a}(C═O)R^{b}, halogen, where R^{a} and R^{b} are independently of each other selected from —H, —(C1-C10)alkyl, —(C1-C10)alkenyl, —(C1-C10)alkynyl, mono or polyfluorinated (C1-C10)alkyl, —(C3-C10)cycloalkyl, —(C3-C10)heterocycloalkyl, -aryl, —(C1-C10)alkylaryl, —(C1-C10)alkylheteroaryl, —(C1-C10)alkyl-heterocycloalkyl or a combination thereof, or R⁵ and R⁷ form together a bond.
(v) when A¹ is —(C═O)—; B¹ and B² form together —C═C—, R³ and R⁵ form together a bond; A³ is —N(R′)— where R⁷ and R′ form together a bond, then A² is not —CH₂—.

The present invention further relates to the compound of the present invention for use in a method for treating and/or preventing cancers presenting tumor-initiating cells.

The present invention further provides a pharmaceutical composition comprising a therapeutically effective amount of the compound of the present invention and pharmaceutically acceptable carrier.

In addition, the present invention also relates to the use of the compound of the invention as antibacterial agent, antifungal agent, pesticide agent and/or herbicide agent.

### BRIEF DESCRIPTION OF FIGURES

FIG. 1 shows short term, dose response for the negative control compound.

FIG. 2 shows short term dose response for the compound of Example 4.

FIG. 3 shows cell loss—Glucose (HepG2 cells); dashed lines indicates significant cut-off from vehicle control (used to calculate minimum effective concentration; MEC) and filled diamond are mean data points for each concentration±standard.

FIG. 4 shows cell loss—Galactose (HepG2 cells); dashed lines indicates significant cut-off from vehicle control (used to calculate minimum effective concentration; MEC) and filled diamond are mean data points for each concentration±standard.

FIG. 5 shows cell loss—Glucose (U-87 MG cells); dashed lines indicates significant cut-off from vehicle control (used to calculate minimum effective concentration; MEC) and filled diamond are mean data points for each concentration±standard.

FIG. 6 shows cell loss—Galactose (U-87 MG cells); dashed lines indicates significant cut-off from vehicle control (used to calculate minimum effective concentration; MSG) and filled diamond are mean data points for each concentration±standard.

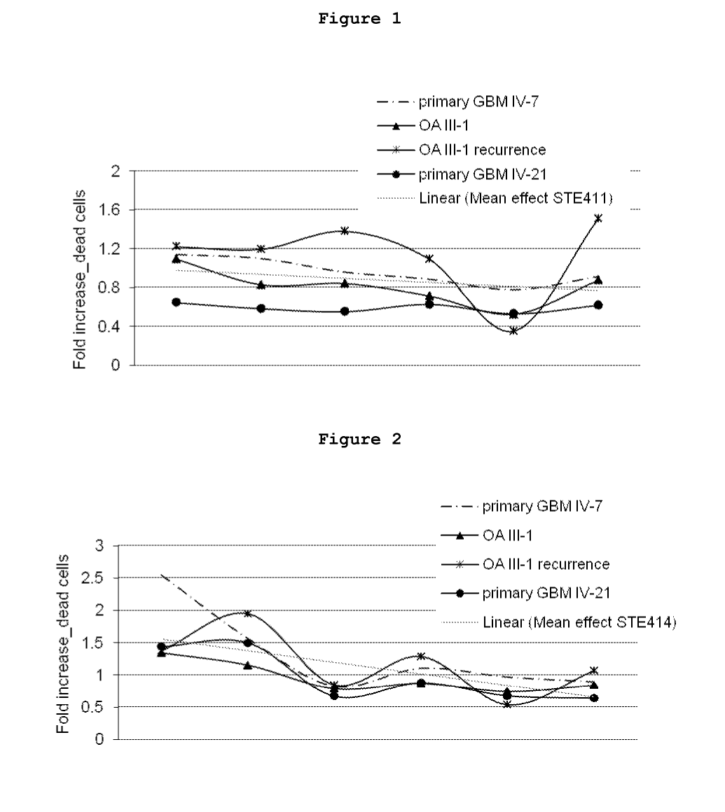

### DETAILED DESCRIPTION OF THE INVENTION

Although methods and materials similar or equivalent to those described herein can be used in the practice or testing of the present invention, suitable methods and materials are described below. All publications, patent applications, patents, and other references mentioned herein are incorporated by reference in their entirety. The publications and applications discussed herein are provided solely for their disclosure prior to the filing date of the present application. Nothing herein is to be construed as an admission that the present invention is not entitled to antedate such publication by virtue of prior invention. In addition, the materials, methods, and examples are illustrative only and are not intended to be limiting.

In the case of conflict, the present specification, including definitions, will control. Unless defined otherwise, all technical and scientific terms used herein have the same meaning as is commonly understood by one of skill in art to which the subject matter herein belongs. As used herein, the following definitions are supplied in order to facilitate the understanding of the present invention.

As used herein, the term “comprise” is generally used in the sense of include, that is to say permitting the presence of one or more features or components.

As used in the specification and claims, the singular form “a”, “an” and “the” include plural references unless the context clearly dictates otherwise.

As used herein the terms “subject” or “patient” are well-recognized in the art, and, are used interchangeably herein to refer to a mammal, including dog, car, rat, mouse, monkey, cow, horse, goat, sheep, pig, camel, and, most preferably, a human. In some embodiments, the subject is a subject in need of treatment or a subject with a disease or disorder, such as cancer presenting tumor initiating cells, preferably glioma and/or cancer presenting glioma initiating cells. However, in other embodiments, the subject can be a normal subject or a subject who has already undergone a treatment, such as for example a prior removal of tumor bulk, for example a tumor glioma bulk. The term does not denote a particular age or sex. Thus, adult, children and newborn subjects, whether male or female, are intended to be covered.

As used herein, the terms “the compound of formula I” or “the compound of the invention” or “the pharmaceutical composition of the invention” also include pharmaceutically acceptable salts or solvates thereof.

The term “alkyl” or “(C1-10) alkyl” used alone or in combination with other groups should be understood to include straight chain and branched hydrocarbon groups having from 1 to 10, preferably 1 to 6 carbon atoms. Alkyl groups may be optionally substituted with one or more substituents. Non-limiting examples of suitable (C1-10)alkyl groups include methyl, ethyl, n-propyl, i-propyl, n-butyl, i-butyl, t-butyl, n-pentyl, n-hexyl.

The term “mono- or polyfluoroalkyl” or “(C1-C10)fluoroalkyl” should be understood as a linear or branched alkyl chain substituted with one or several fluorine atoms. Non-limiting examples of such groups are fluoromethyl, trifluoromethyl, trifluoroethyl, 3-fluoropropyl, 4-fluorobutyl.

The term “alkenyl” refers to an alkyl group containing at least one carbon-carbon double bond. Alkenyl groups may be optionally substituted with one or more substituents.

The term “alkynyl” refers to an alkyl group containing at least one carbon-carbon triple bond. Alkynyl groups may be optionally substituted with one or more substituents.

The optional substituents of alkyl, alkenyl and alkynyl groups may be the same or different, and are independently selected from H, (C1-10)alkyl, —OR′, —NR′R″, —OC(═O)OR′, —C(═O)OR′, —C(═O)NR′R″, —CF₃, —CN, —NO₂ or halogen, wherein R′ and R″ represent H or (C1-10)alkyl.

The term “branched” should be understood to represent a linear straight chain hydrocarbon group having one or more lower alkyl groups such as methyl, ethyl or propyl, attached to it.

The term “halogen” (or “hal”) should be understood to include fluoro, chloro, bromo, iodo, preferably fluoro and chloro, most, preferably fluoro.

The term “cycloalkyl” unless defined otherwise refers to a saturated monocyclic or bicyclic ring system having 3 to 10, preferably 3, 4, 5, 6 or 10, more preferably 3, 4, 5 or 6 ring atoms. Preferred cycloalkyl groups include cyclopropyl, cyclobutyl, cyclopentyl, cyclohexyl, norbornyl and the like.

The term “alkyl-cycloalkyl” (or “—(C1-C10)alkyl-cycloalkyl”) refers to a radical wherein alkyl (or (C1-10)alkyl) and cycloalkyl have the meanings as defined above. Illustrative examples of an alkyl-cycloalkyl group or radical include 1-cyclopropylmethyl, 1-cyclopentylmethyl, bis-azetidine spirocycles such as 2,6-diazaspiro[3.3]heptane, azetidine-thietane spirocycles such as 2-thia-6-azaspiro[3.3]heptane and azetidine-oxetane spirocycles such as 2-oxa-6-azaspiro[3.3]heptane.

The term “heterocycloalkyl” unless defined otherwise refers to a cycloalkyl group as defined above, wherein one or more of the atoms in the ring system, preferably 1 to 3 is/are replaced by heteroatoms chosen from the group consisting of O, S, and N. Preferred heterocycloalkyl include oxetane, thioxetane, azetidine, tetrahydrofurane, tetrahydropyrane, pyrrolidine, piperidine, piperazine, oxazines, such as morpholine, thiamines and the like.

The term “alkyl-heterocycloalkyl” refers to a radical wherein alkyl and heterocycloalkyl have the meanings as defined above. Illustrative examples of an alkyl-heterocycloalkyl group are oxetan-3-ylmethyl, tetrahydrofuran-2-ylmethyl and 2-oxa-6-azaspiro[3.3]heptanylmethyl.

The term “aryl” unless defined otherwise should be understood to include a monocyclic or bicyclic, aromatic ring system having 5 to 10, preferably 5, 6 or 10, more preferably 5 or 6 ring atoms. Non-limiting examples of suitable aryl groups include phenyl, (1- or 2-)naphthyl or tetraline groups, most preferably phenyl groups.

The term “alkyl-aryl” (or “(C1-10)alkylaryl”) refers to a radical wherein alkyl (or (C1-10) alkyl) and aryl have the meanings as defined above. Illustrative examples of an alkyl-aryl group or radical include benzyl, 2-phenylethyl, 3-phenylpropyl, 4-phenylbutyl, 1-naphthylmethyl.

The term “alkenyl-aryl” (or “(C1-10)alkenylaryl”) refers to a radical wherein alkenyl (or (C1-10)alkenyl) and aryl have the meanings as defined above, A particular example of an alkenyl-aryl group or radical is vinyl benzene.

The term “heteroaryl” unless defined otherwise should be understood to include an aromatic ring system of 5 to 10, preferably 5, 6 or 10, more preferably 5 or 6 ring atoms, in which one or more of the atoms in the ring system is/are atoms other than carbon, for example nitrogen, oxygen or sulfur, Preferably, the aromatic heteroaryl is a 5- or 6-membered aromatic ring having 1 to 3 heteroatoms selected from N, O, S, preferably N and O, and benzo-fused derivatives thereof. Examples of suitable 6-membered heteroaryl groups include pyridine, pyrimidine, pyrazine, pyridazine and the like. Examples of useful 5-membered aromatic heteroaryls include furan, pyrrole, triazole, thiazole, isothiazole, imidazole, pyrazole, oxazole and isoxasole. Useful bicyclic groups are benzo-fused ring systems derived from the aromatic heteroaryls named above, e.g., quinoline, phthalazine, quinazoline, benzofuran, phthaleimide and indole. Most preferred examples are pyridine, pyrimidine, oxazole, thiazole, imidazole, triazole, pyrazole, and phthaleimide.

The cyclic groups aryl, heteroaryl, cycloalkyl and heterocycloalkyl can be substituted with one or more substituents R′, preferably one, two or three substituents R′, which may be the same or different, and are independently selected from H, (C1-10)alkyl, —OR″, —NR″R″′, —OC(═O)R″, —C(═O)OR″, —C(═O)NR″R″′, —CH═CHC(═O)OR″, —CF₃, —CN, —NO₂, or halogen, wherein R″ and R″′ represent H or (C1-10)alkyl. The substituents R′ may be in any position. For example in monosubstituted phenyl residues the substituent can be located in the ortho-position, the meta-position or the para-position, preferably in the para-position. If phenyl is substituted twice, the substituents can be in the ortho/meta-position (all possibilities), the ortho/para-position, the ortho/ortho-position, the meta/meta-position, or the meta /para-position, In substituted heteroaryl or heterocycloalkyl groups, the substituent may reside on a carbon atom or a heteroatom, e.g. N-methyl pyrrole and the like.

The terms “bicycloalkyl” and “heterobicycloalkyl” should be understood as fragments that contain two or more small-, medium- and large-scale rings fused together to form a bicyclic fragment. Such rings can contain one or several heteroatoms (O, N, S, etc, atoms) or unsaturations (CH═CH, —(C═O)—, —CC—, etc.). The two rings can be fused in a spiro fashion (on carbon atom in common) or bridged (sharing two or n atoms, with a bridge containing (n-2) atoms). Preferably, if one of the rings is an aromatic ring, it will be a 5 to 6 membered aryl or heteroaryl as described above. Preferably, at least one of the cycloalkyl or heterocycloalkyl units contains 3-12 atoms. The bicyclic structure thus formed can be additionally substituted with one or more substituents R′, preferably one, two or three substituents R′, which may be the same or different, and are independently selected from H, (C1-10)alkyl, —OR″, —NR″R″′, —OC(═O)R″, —C(═O)OR″, —C(═O)NR″R″′, —CH═CHC(═O)OR″, —CF₃, —CN, —NO₂, or halogen, wherein R″ and R″′ represent H or (C1-10) alkyl. The substituents R′ may be in any position.

Applicants have found that tumor-initiating cells (TICs), such as glioma-initiating cells (GICs), (more specifically the FL1⁺ cell population as used herein) do produce their energy, divide, and survive using the aerobic pathway (TCA cycle/oxidative phosphorylation—electron transport chain). The Applicants have also found that for example the glioma-initiating cells (GICs) have a different metabolism than others glioma cells (cancer cells) from the tumor bulk, which preferentially uses the aerobic glycolysis (Warburg's effect). Indeed the Applicants made an interesting finding that FL1⁺ cells (CICs) are enriched for NADH, for active mitochondria, and active LD. Furthermore, FL1⁺ cells have lower levels of lactate compared to FL1⁰ cells, suggesting that FL1⁺ cells might preferentially used the aerobic-mitochondria pathway to produce ATP.

The identification of the compounds useful in the treatment of cancers presenting tumor-initiating cells implies the use of a reliable selection method to identify, isolate and characterize the whole cancer-initiating cells (CICs) or tumor-initiating cells (TICs) reservoir and a specific and robust method to test the compounds.

Different developed approaches as recently described in international patent applications no PCT/IB2008/054872 (SO 2009/066258) and no PCT/IB2010/052237 (WO 2010/134039), which are incorporated herein by reference, were used to isolate and enrich for a subpopulation of cells showing self-renewing and tumor-initiating properties.

Both methods lie on primary cell cultures derived from human specimen and rely on simple and robust phenotypic characteristics of tumor cells to trace and distinguish viable TICs (referred as FL1⁺ cells) from the non tumorigenic cells (referred as FL1⁰ cells) independently of any cell surface marker.

The efficacy of a compound in decreasing and/or eradicating the tumor-initiating cells (e.g. recurrence of the cancer initiating cells) may be assayed by detecting the presence of initiating cells in a cell sample after treatment with the compound according to the present invention, for example by a method as described in PCT/IB2008/054872 and PCT/IB2010/052237, i.e. comprising the following steps:

a) Providing a cancer stem cell sample which was treated by a compound or a method according to the invention;

b) Incubating the treated stem cell sample in a stem cell culture medium for an incubation period without treatment;

c) Selecting the viable cell population from the stem cell sample incubated under step (b);

d) Measuring the mean level of autofluorescence on the viable cell population isolated, under step (c) by detecting, by fluorescence activated cell sorting, cells presenting autofluorescence emission in the FL1 channel upon laser beam excitation at a wavelength of or about 488 nm;

e) Isolating cells by fluorescence activated cell sorting cell which have a specific morphology (high FSC and low/middle SSC) and present autofluorescence emission in the FL1 channel upon laser beam excitation at a wavelength of or about 488 nm of the viable cell population isolated under step (c);

f) Isolating cells by fluorescence activated cell sorting which have a specific morphology (low/middle FSC and middle/high SSC), do not present autofluorescence emission in the FL1 channel under step (c) and present a slight positive shift in the cell fluorescence emission in the FL3 and/or FL1 channel upon laser beam excitation of the viable cell population isolated under step (c);

g) Calculating the percentage of autofluorescent viable cells by comparing the mean level of autofluorescence in the cancer stem cell sample provided under step (a) and the mean level of autofluorescence measured under step (d);

h) Calculating the percentage of the cell death by comparing the number of initial cells present in the cancer stem cell, sample provided under step (a) and the resulting viable cell population isolated under step (c);

i) Calculating the percentage of viable FL1⁺ cells by comparing the number of initial FL1⁺ cells present in the cancer stem cell sample provided under step (a) and the resulting viable FL1⁺ cell population isolated under step (e);

j) Calculating the percentage of viable FL1⁰ cells by comparing the number of initial FL1⁰ cells present in the cancer stem cell sample provided under step (a) and the resulting viable FL1⁰ cell population isolated under step (f);

k) Detecting spherogenicity of the cell populations detected under steps (e) and (f).

l) Determining the activity of the agent through its ability to inhibit cancer stem cells recurrence.

The in vitro and in vivo phenotypic and behaviour differences between FL1⁺ and FL1⁰ tumor cell populations was supported by further characterization demonstrating that FL1⁺ cells are enriched for stemness-related genes, are multipotent, can generate FL1⁰ cells and are responsible for maintaining the long-term self-renewal capacity overtime. Because FL1⁰ derived cultures do not yield any FL1⁺ cell, it provides further-evidence that FL1⁰ cells are derived from the FL1⁺ population, remain viable for several passages, but are unable to reacquire autofluorescent properties once they have switched from the FL1⁺ toward the FL1⁰ state.

Applicants found that compounds, which, target the oxidative cellular energy production process, demonstrate a reliable and long-lasting efficacy to eradicate tumor-initiating cells. Compounds which prevents NADH from being converted into cellular ATP at the mitochondrial III and induces the formation of H₂O₂ generation might therefore be considered as novel and specific therapeutic strategy against tumor-initiating cells.

Applicants also demonstrate that blocking the production of energy generated by the aerobic pathway is sufficient for killing the whole tumor-initiating cell population (the killing is done not by apoptosis, but by starving tumor-initiating cells, i.e. blocking the production of energy in tumor-initiating cells). Compounds, which can be inhibitors, interfering with the electron transport chain such as the one of mitochondria at the level of the complex III are demonstrating an exceptional capacity to kill every tumor-initiating cells in vitro and in vivo. As the inhibition of complex III results in large production of reactive oxygen species (ROS) and free radicals, it is likely that the tumor-initiating cells are also killed by the accumulation of ROS or the saturation of the detoxification system.

Applicants found that the compounds of the present invention are inhibitors of the activity of Complex (III) of the mitochondrial electron transport chain. This finding provides the application of the compounds of the present invention not only in treatment and prevention of cancers presenting tumor-initiating cells, but also in many other fields where the inhibition of the activity of Complex (III) of the mitochondrial electron transport chain is beneficial. This includes, for example, antibacterial, antifungal, pesticide and herbicide applications.

The present invention aims to provide a compound of formula I

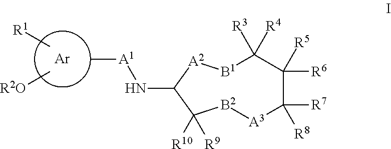

or a pharmaceutically acceptable salt or solvate thereof, wherein

Ar is selected from (C5-C10)aromatic ring or (C5-C10) heteroaromatic ring where one or more of the carbon atoms in the ring system are replaced by heteroatoms selected from the group consisting of O, S, and N;

R¹ is selected from H, —(C1-C10)alkyl, -aryl, —OH, —O—(C1-10)alkyl, —O-aryl, —NH₂, —NH(CHO), —NH(C═O)—(C1-10)alkyl, halogen, —NO₂, —C(═O)OH, —C(═O)O(C1-10)alkyl, —CF₃, —NH(C═O) (CF₃(OMe))Ph;

R² is selected from H, —(C1-10)alkyl, aryl, benzyl, —(C═O)(CF₃(OMe))Ph;

R³ is selected from —H, —(C1-10)alkyl, —CF₃ or can form a bond with R⁵;

R⁴, R⁹ and R¹⁰ are independently of each other selected from —H, —(C1-10)alkyl, —CF₃;

A¹, A² and A³ are independently of each other selected from: —(C═O)—, —(C═S)—, —(S═O)—, —(S(═O)₂)—, —(CH₂)—, —(C(CH₃)₂)—, —CH(CF₃)—, —CHF—, —(C(CH₂OCH₂))—, —(C(CH₂SCH₂))—, —N(R^{a})—, —(CR′R″)—, —CH₂—CH₂—, —(C(CH₃)₂)—, where R^{a} is selected from —H, —(C1-C10)alkyl, —(C1-C10)alkenyl, —(C1-C10)alkynyl, mono or polyfluorinated (C1-C10)alkyl, —(C3-C10)cycloalkyl, —(C3-C10)heterocycloalkyl, -aryl, —(C1-C10)alkylaryl, —(C1-C10)alkylheteroaryl, —(C1-C10)alkyl-heterocycloalkyl, and where R′ and R″ are independently of each other selected from —H, —(C1-C10)alkyl, -halogen, —CF₃;

B¹ and B²:
(i) are independently of each other selected from —O—, —S—, —NH—, —CH₂—, or
(ii) form together —CH—CH— producing a bicyclo(3,4,0)-nonane unit
(iii) form, together —C═C— producing a bicycle
(iv) form together a —N—N— or —CH—N— fragment producing a diazabycycle or azabicycle

R⁵, R⁶, R⁷ and R⁸ are selected from:
(i) independently of each other, —R^{a}, —OR^{a}, —O(C═O)R^{a}, —C(═O)R^{a}, —C(═O)OR^{a}, —NR^{a}R^{b}, —NR^{a}(C═O)R^{b}, halogen, where R^{a} and R^{b} are independently of each other selected from —H, —(C1-C10)alkyl, —(C1-C10)alkenyl, —(C1-C10)alkynyl, mono or polyfluorinated (C1-C10)alkyl, —(C3-C10)cycloalkyl, —(C3-C10)heterocycloalkyl, -aryl, —(C1-C10)alkylaryl, —(C1-C10)alkylheteroaryl, —(C1-C10)alkyl-heterocycloalkyl or a combination thereof, or;
(ii) R⁵ and R⁷ form together a bond AND R⁶ and R⁸ are independently of each other selected from —R^{a}, —OR^{a}, −O(C═O)R^{a}, —C(═O)R^{a}, —C(═O)CR^{a}, —NR^{a}R^{b}, —NR^{a}(C═O)R^{b}, halogen, where R^{a} and R^{b} are independently of each other selected from —H, —(C1-C10)alkyl, —(C1-C10)alkenyl, —(C1-C10)alkynyl, mono or polyfluorinated (C1-C10)alkyl, —(C3-C10)cycloalkyl, —(C3-C10)heterocycloalkyl, -aryl, —(C1-C10)alkylaryl, —(C1-C10)alkylheteroaryl, —(C1-C10)alkyl-heterocycloalkyl or a combination thereof, or;
(iii) R⁵ and R⁷ represent independently of each other, —R^{a}, —OR^{a}, —O(C═O)R^{a}, —C(═O)R^{a}, —C(═O)OR^{a}, —NR^{a}Rb, —NR^{a}(C═O)R^{b}, halogen, where R^{a} and R^{b} represent independently of each other —H, —(C1-C10)alkyl, —(C1-C10)alkenyl, —(C1-C10)alkynyl, mono or polyfluorinated (C1-C10)alkyl, -aryl AND R⁶ and R⁸ form together a (C3-C10)cycloalkyl or (C3-C10)heterocycloalkyl substituted by R^{c} and R^{d} where R^{c} and R^{d} represent independently of each other —H, —(C1-C10)alkyl, —(C1-C10)alkenyl, —(C1-C10)alkynyl, mono or polyfluorinated (C1-C10)alkyl, -aryl, —OH, —O—(C1-C10)alkyl, —O—aryl, —O(C═O)—(C1-C10)alkyl, —O(C═O)-aryl, halogen, or;
(iv) R⁵ and R⁷ form together a bond AND R⁶ and R⁸ form together a (C3-C10)cycloalkyl or heterocycloalkyl substituted by R^{c} and R^{d} where R^{c} and R^{d} represent independently of each other —H, —(C1-C10)alkyl, —(C1-C10)alkenyl, —(C1-C10)alkynyl, mono or polyfluorinated (C1-C10)alkyl, -aryl, —OH, —O—(C1-C10)alkyl, —O-aryl, —O(C═O)—(C1-C10)alkyl, —O(C═O)-aryl, halogen, or;
(v) R⁵ and R⁷ form together a bond AND R⁶ and R⁸ form together an (C6-C10)aryl or (C5-C10)heteroaryl substituted by R^{c} and R^{d} where R^{c} and R^{d} represent independently of each other —H, —(C1-C10)alkyl, —(C1-C10)alkenyl, —(C1-C10)alkynyl, mono or polyfluorinated (C1-C10)alkyl, -aryl, —OH, —O—(C1-C10) alkyl, —O-aryl, —O(C═O)—(C1-C10)alkyl, —O(C═O)-aryl, halogen, or;
(vi) when B¹ and B² form together —C═C—, R³ and R⁵ form together a bond AND A³ is —C(R′R″)— or —N(R′)— AND R⁷ and R′ form together a bond, where R′ and R″ are independently of each other selected from —H, —(C1-C10)alkyl, -halogen, —CF₃;
   with the provisos that
1) when A¹, A² and A³ are —C(═O)— AND B¹ and B² are —O— AND R³, R⁵, R⁷ and R⁹ are —H, the compound of formula I excludes the compound of formula II

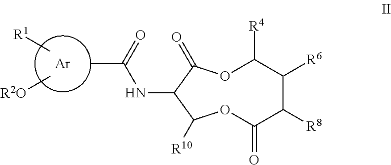

wherein
   Ar is an aromatic ring selected from benzene, pyridine, indazole, benzotriazole, benzymidazole or pyrazolothiophene;
   R¹ is selected from H, —C(1-10)alkyl, —O(C1-C10)alkyl, —Cl, —NO₂, —NH₂, —NHC(—O)H, —NH(C═O)—C(1-10)alkyl, —N[(C1-C10)alkyl]₂, —N[C(1-10)alkyl]C(—O)C(1-10)alkyl, —NH(C═O) (CF₃(OMe))Ph;
   R² is selected from H, —C(1-10)alkyl, phenacyl, benzyl, benzoyl, —(C═O) (CF₃(OMe))Ph;
   R⁴ and R¹⁰ are independently of each other selected from —H or —(C1-C10)alkyl;
   R⁶ and R⁸ are independently of each other selected from —H, —(C1-C10)alkyl , —OH, —O(C1-C10)alkyl, —OC(═O)(C1-C10)alkyl, —O—benzyl, —OC(—O)aryl, —OCO(CH₃)—CHCH₃, —CH₂C₆H₅, or —O(C═O) benzyl wherein —(C1-C10)alkyl, —O(C1-C10)alkyl, —OC(—O) (C1-C10)alkyl, —O—benzyl, —OC(—O)aryl and —O(C═O)benzyl are optionally substituted with —OH, -halogen, —NO₂, —CO₂H;
2) when A¹, A² and A³ are —C(═O)— AND B¹ and B² are —O— AND R³ and R⁹ are —H AND R⁵ and R⁷ form together a bond AND R⁶ and R⁸ form together an aryl ring, the compound of formula I excludes the compound of formula III

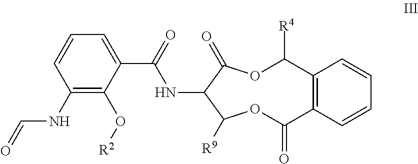

wherein
   R², R⁴ and R⁹ are independently of each other selected from —H or —(C1-C10)akyl;
3) when A¹ is —C(═O)— AND A² is —CH₂— AND A³ is —C(R′R″)— AND R⁹ and R¹⁰ are —H AND B¹ and B² form together a —C═C— AND R³ and R⁵ and R⁷ and R′ form together a bond, the compound of formula I excludes the compound of formula IV

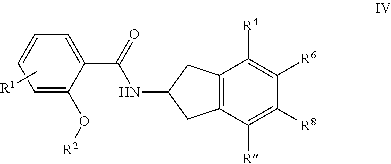

wherein
   R″, R⁴, R⁶ and R⁸ are independently of each other selected from —H or —(SO₂)—R^{e} where R^{e} is selected from -halogen, —(C1-C10)alkyl, -cycloalkyl, heterocycloalkyl, -aryl, -heteroaryl, —NH₂, —OH or combination thereof;
   R¹ is selected from H, —(C1-C10)alkyl, -aryl, —OH, —O—(C1-10)alkyl, —O—aryl, —NH₂, —NH(CHO), —NH(C═O)—(C1-10)alkyl, halogen, —NO₂, —C(═O)OH, —C(═O)O(C1-10)alkyl, —CF₃, —NH(C═O) (CF₃(OMe))Ph;
   R² is selected from H, —(C1-10)alkyl, aryl, benzyl, —(C═O) (CF₃(OMe))Ph;
4) when A¹ is —C(—O)— AND A³ is —N(R′)— AND R⁹ and R¹⁰ are —H AND B¹ and B² form together a —CH—N— fragment AND R³, R⁶ and R⁷ are —H, the compound of formula I excludes the compound of formula V

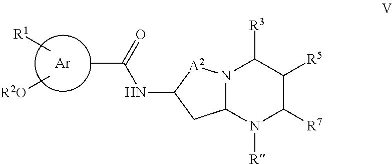

wherein
   A² is selected from —CH₂— or —C(═O)—
   Ar is selected from (C5-C10)aromatic ring or (C5-C10)heteroaromatic ring;
   R¹ is selected from H, —(C1-C10)alkyl, -aryl, —OH, —O—(C1-10)alkyl, —O—aryl, —NH₂, —NH(CHO), —NK(C═O)—(C1-10)alkyl, halogen, —NO₂, —C(═O)OH, —C(═O)O(C1-10)alkyl, —CF₃, —NH(C═O) (CF₃ (OMe))Ph;
   R² is selected from H, —(C1-10)alkyl, aryl, benzyl, —(C═O) (CF₃(OMe))Ph;
   R³ is selected from —H, —(C1-10)alkyl, —CF₃ or can form a bond, with R⁵;
   R⁵ and R⁷ are selected from independently of each other, —R^{a}, —OR^{a}, —O(C═O)R^{a}, —C(═O)R^{a}, —C(═O)OR^{a}, —NR^{a}R^{b}, —NR^{a}(C═O)R^{b}, halogen, where R^{a} and R^{b} are independently of each other selected from —H, —(C1-C10)alkyl, —(C1-C10)alkenyl, —(C1-C10)alkynyl, mono or polyfluorinated (C1-C10)alkyl, —(C3-C10)cycloalkyl, —(C3-C10)heterocycloalkyl, -aryl, —(C1-C10)alkylaryl, —(C1-C10)alkylheteroaryl, —(C1-C10)alkyl-heterocycloalkyl or a combination thereof, or R⁵ and R⁷ form together a bond.
5) when A¹ is —(C═O)—; B¹ and B² form together —C═C—, R³ and R⁵ form together a bond; A³ is —N(R′)— where R⁷ and R′ form together a bond, then A² is not —CH₂—.

Preferably Ar is selected from (C5-C6)aromatic ring or (C5-C6)heteroaromatic ring where one or more of the carbon atoms in the ring system are replaced by heteroatoms selected from the group consisting of O, S, and N. More preferably Ar is (C5-C6)aromatic ring.

Preferably A¹ is —(CO)—.

Preferably A² and A³ are —C(O)—.

Preferably B¹ and B² are independently of each other selected from —O—, —S—, —NH—, —CH₂—, with the proviso that at least one of B¹ and B² is not —O—.

More preferably B¹ and B² are —O—.

Preferably R⁶ and R⁸ form together a (C3-C10) cycloalkyl or (C3-C10) heterocycloalkyl substituted by R^{c} and R^{d} where R^{c} and R^{d} are independently of each other selected from —H, —(C1-C10)alkyl, —(C1-C10)alkenyl, —(C1-C10)alkynyl, mono or polyfluorinated (C1-C10)alkyl, -aryl, —OH, —O—(C1-C10)alkyl, —O—aryl, —O(C═O)—(C1-C10)alkyl, —O(C═O)-aryl, halogen.

Preferably R⁵, R⁸ and R¹⁰ are —H.

In an embodiment of the present invention, preferably R⁵ and R⁷ form a bond together. According to this embodiment, preferably A² and A³ are —C(═O)— and R¹⁰ is H,

Preferably R⁶ and R⁸ form together a (C3-C10)cycloalkyl ring or a (C3-C10)heterocycloalkyl ring substituted with R^{c} and R^{d}, where R^{c} and R^{d} represent independently of each other —H, —(C1-C10)alkyl, —(C1-C10)alkenyl, —(C1-C10)alkynyl, mono or polyfluorinated (C1-C10)alkyl, -aryl, —OH, —O—(C1-C10)alkyl, —O-aryl, —O(C═O)—(C1-C10)alkyl, —O(C═O)-aryl, halogen,

More preferably R⁶ and R⁸ form together a (C6-C10)aryl group or a (C5-C10)heteroaryl group substituted with R^{c} and R^{d}, where R^{c} and R^{d} represent, independently of each other —H, —(C1-C10)alkyl, —(C1-C10)alkenyl, —(C1-C10)alkynyl, mono or polyfluorinated (C1-C10)alkyl, -aryl, —OH, —O—(C1-C10)alkyl, —O-aryl, —O(C═O)—(C1-C10)alkyl, —O(C═O)-aryl, halogen.

More preferably:

R¹⁰ is —H;

A² and A³ are independently of each other selected from —(C═O)—, —(C═S)—, —(S═O)—, —(S(═O)₂)—, —(CH₂)—, —(C(CH₃)₂)—, —CH(CF₃)—, —CHF—, —(C(CH₂OCH₂))—, —(C(CH₂SCH₂))—, —N(R^{a})—, —(CR′R″)—, —CH₂— CH₂—, —(C(CH₃)₂)—, where R′ and R″ are independently of each other chosen from —H, —(C1-C10)alkyl, -halogen, —CF₃ with the proviso that A² and A³ cannot be —C(═O)— at the same time.

In another embodiment of the invention, preferably

R¹⁰ is —H;

B¹ and B² form together a —CH—CH—, —C═C—, —CH—N— or —N—N— fragment forming respectively a bicyclic scaffold.

More preferably B¹ and B² form together a —CH—CH— fragment forming a (3,4,0) bicycle.

More preferably B¹ and B² form together a —C═C— providing a bicyclic scaffold; R³ and R⁵ form, together a bond; A³ represents —C(R′R″)— or —N(R′)— and R⁷/R′ form together a bond.

In a further embodiment of the invention, preferably A² and A³ are independently of each other selected from —(C═O)—, —(C═S)—, —(S═O)—, —(S(═O)₂)—, —(CH₂)—, —(C(CH₃)₂)—, —CH(CF₃)—, —CHF—, —(C(CH₂OCH₂))—, —(C(CH₂SCH₂))—, —N(R′)—, —(CR′R″)—, —CH₂—CH₂—, —(C(CH₃)₂)—, where R′ and R″ are chosen from —H, —(C1-C10) alkyl, halogen, —CF₃, with the proviso that A² and A³ cannot be —C(═O)— at the same time.

Preferably R⁵, R7 and R¹⁰ are —H.

In a further embodiment of the invention, preferably B¹ and B² are independently of each other selected from —O—, —S—, —NH—, —CH₂—, with the proviso that at least one of B¹ and B² is not —O—.

Preferably R⁵, R⁷ and R¹⁰ are —H.

In another embodiment of the invention, preferably A² and A³ are —C(═O)—; B¹ and B² are —O—; R⁵, R⁷ and R¹⁰ are —H; with the proviso that at least one of the side chains R⁶ and R⁸ contains a —(C1-C10)alkyl-heterocycloalkyl group or a mono/polyfluorinated (C1-C10)alkyl;

In one embodiment, the central motif of the compounds of the invention is a nine-membered bis-lactone.

In another embodiment, the central motif of the compounds of the invention is a nine-membered cycloalkyl ring or heterocycloalkyl ring containing 1 or 2 heteroatoms and 0, 1, 2 or 3 insaturations.

In yet another embodiment, the central motif of the compounds of this invention is a (3,4,0)-bicycle containing 0, 1 or 2 heteroatoms;

In yet another embodiment, the central motif of the compounds of this invention is an indenyl or cyclopentane-pyridinyl fragment.

In a preferred embodiment, the linker between the aminosalicylic group and the 9-membered ring is an amide.

In another preferred embodiment, the substituents —OR² and A¹ on the aromatic/heteroaromatic group Ar are in ortho positions.

According to another preferred embodiment of the present invention, the compound of the invention is of formula VI

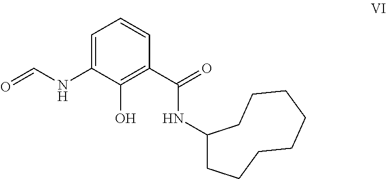 [SMILES: O=CNc1cccc(C(=O)NC2CCCCCCCC2)c1O]

“Compound” and “compounds” as used herein refers to a compound encompassed by the generic formulae disclosed, in the present invention, any subgenus of those generic formulae, and any forms of the compounds specified by the generic and subgeneric formulae, such as a pharmaceutically acceptable salt or solvate. Unless specified otherwise, it is further understood that all isomers, including enantiomers, stereoisomers, rotamers, tautomers and racemates of the compound(s) of the invention are contemplated as being part of this invention. The invention includes stereoisomers in optically pure form and in admixture, including racemic mixtures. Isomers can be prepared using conventional techniques, either by reacting optically pure or optically enriched starting materials or by separating isomers of a compound of formula I.

“Racemates” refers to a mixture of enantiomers.

“Stereoisomer” or “stereoisomers” refer to compounds that differ in the chirality of one or more stereocentres. Stereoisomers include enantiomers and diastereomers. The compounds of this invention may exist in stereoisomeric form if they possess one or more asymmetric centres or a double bond with asymmetric substitution and, therefore, can be produced as individual stereoisomers or as mixtures. Unless otherwise indicated, the description is intended to include individual stereoisomers as well as mixtures. The methods for the determination of stereochemistry and the separation of stereoisomers are well-known in the art (see discussion in Chapter 4 of Advanced Organic Chemistry, 4th ed., J. March, John Wiley and Sons, New York, 1992).

“Tautomer” refers to alternate forms of a compound that differ in the position of a proton, such as enol-keto and imine-enamine tautomers, or the tautomeric forms of heteroaryl groups containing a ring atom attached to both a ring —NH-moiety and a ring ═N-moiety such as pyrazoles, imidazoles, benzimidazoles, triazoles, and tetrazoles.

A skilled person will know that, if a compound of the invention contains charged group, a suitable counterion will be derived from an organic or inorganic acid. Such counterions include halide (such as chloride, bromide, fluoride, iodide), sulfate, phosphate, acetate, succinate, citrate, lactate, maleate, fumarate, palmitate, chelate, glutamate, glutarate, tartrate, stearate, salicylate, methanesulfonate, benzenesulfonate, sorbate, picrate, benzoate, cinnamate, and the like. If the polar moiety is a negatively charged group, a suitable counterion will be selected from sodium, ammonium, barium, calcium, copper, iron, lithium, potassium and zinc, and the like.

According to the present invention, pharmaceutically acceptable salts are produced from acidic inorganic or organic compounds, or alkaline inorganic or organic compounds.

As used herein, the phrase “pharmaceutically acceptable salt” refers to a salt that retains the biological effectiveness of the free acids and bases of a specified compound and that is not biologically or otherwise undesirable. The pharmaceutically acceptable salts of the compounds of formula (I) are acid addition salts with pharmaceutically acceptable acids.

A desired salt may be prepared by any suitable method known in the art, including treatment of the free base with an inorganic acid, such as hydrochloric acid, hydrobromic acid, sulphuric acid, nitric acid, phosphoric acid, and the like, or with an organic acid, such as formic acid, acetic acid, maleic acid, succinic acid, mandelic acid, maleic acid, fumaric acid, malonic acid, pyruvic acid, oxalic acid, glycolic acid, salicylic acid; a pyranosidyl acid, such as glucuronic acid or galacturonic acid; an alpha-hydroxy acid, such as citric acid or tartaric acid; an amino acid, such as aspartic acid or glutamic acid; an aromatic acid, such as benzoic acid or cinnamic acid; a sulfonic acid, such as methanesulfonic acid, p-toluenesulfonic acid or ethanesulfonic acid; or the like.

In the present invention the preferred ammonium salts are derived from hydrochloric, hydrobromic, methanesulfonic, acetic, propionic, benzoic, citric, tartaric, malic, maleic, fumaric, lactic, nitric, and phosphoric or succinic acid.

Generally, the salts are prepared by reacting the free base with stoichiometric amounts or with an excess of the desired salt forming inorganic or organic acid in a suitable solvent or various combinations of solvents. For example, the free base can be dissolved in a mixed aqueous solution of the appropriate acid and the salt recovered by standard techniques, for example, by evaporation of the solution. Alternatively, the free base can be charged into an organic solvent such as a lower alkanol, symmetrical or asymmetrical ethers containing 2 to 10 carbon atoms, an alkyl ester, or mixtures thereof, and the like, and then it is treated with the appropriate acid to form the corresponding salt. The salt is recovered by standard recovery techniques, for example, by filtration of the desired salt from the mixture, or it can be precipitated by the addition of a solvent in which the salt is insoluble and recovered there from.

Examples of suitable inorganic and organic solvents for performing the various reactions include any inorganic or organic solvent that does not adversely affect the reactants or the resulting product, including halogenated solvents such as methylene chloride, chloroform, ether solvents such as diethyl ether, and other solvents such as tetrahydrofuran, dioxane, diglyme, cyclooctane, benzene or toluene, heptane, cyclohexane, aliphatic as well as cycloaliphatic and aromatic hydrocarbon solvents, water, acidified aqueous solutions, mixed organic and inorganic solutions, ethyl acetate, propyl acetate and mixtures thereof.

Also encompassed by the present invention are salts formed from acidic prodrugs, such as phosphates, and alkaline inorganic or organic compounds. Preferred inorganic cations comprised in the salts are lithium, sodium, potassium, rubidium, ammonium, calcium, magnesium, zinc and manganese.

Production of phosphate salts are described in e.g. G. R. Pettit et al. Anti-Cancer Drug Design 16 (2001) 185-193.

Preferred salts also include those formed from acidic prodrugs and organic amines, including, but not limited to, imidazole and morpholine. Alkaline amino acid salts may also be used. The term “amino acids” designates, according to the invention, in particular the [alpha]-amino acids occurring in nature, but moreover also includes their homologues, isomers and derivatives. Enantiomers can be mentioned as an example of isomers. Derivatives can be, for example, amino acids provided with protective groups. Preferred alkaline amino acid are arginine, ornithine, diaminobutyric acid, lysine or hydroxy lysine and especially L-arginine, L-lysine or L-hydroxy lysine; an alkaline dipeptide or a pharmaceutically acceptable alkaline amino acid derivate.

A “pharmaceutically acceptable solvate” or “solvate” refers to an aggregate or physical association of a compound of the present invention with one or more solvent molecules. The solvent may be water or any common organic solvent. In the context of the present invention, preferred solvent molecule is PEG. This physical association involves varying degrees of ionic and covalent bonding, including hydrogen bonding. In certain embodiments, the solvate will be capable of isolation, for example when one or more solvent molecules are incorporated in the crystal lattice of the crystalline solid. “Solvate” encompasses both solution-phase and isolable solvates. Representative solvates include ethanolates, methanolates, and the like. “Hydrate” is a solvate wherein the solvent molecule(s) is/are H₂O.

The present invention also relates to pro-drugs of a compound of formula I that in vivo convert to the compound of formula I as such. Any reference to a compound of formula I is therefore to be understood as referring also to the corresponding pro-drug of the compound of formula I, as appropriate.

For the purposes of the present invention, a “pro-drug” is an entity which either comprises an inactive form of an active drug (parent compound) or includes a chemical group which confers preferred characteristics on the drug. In other words, it concerns a composition, which has the potential of producing a desired physiological effect on cells, but is initially inert (i.e. does not produce said effect), and only after undergoing some modifications becomes physiologically active and produces said physiological effect on cells. In particular, the derivative of the compound of formula I has a chemically or metabolically degradable group, and becomes pharmaceutically active after biotransformation.

Biotransformation of the prodrug or a salt thereof is carried out under physiological conditions (in vivo) and is a result of a reaction with an enzyme, or a body fluid such as gastric acid, blood etc., thus undergoing an enzymatic oxidation, reduction, hydrolysis etc. or a chemical hydrolysis convert into the active parent compound of formula I.

As used herein, the terms “parent compounds” or “active parent, compounds” or “active drugs” are used interchangeably herein to designate the compounds of formula I according to the present invention.

The term, “physiological effect” concerns any effect a drug may have on cells, in order to improve the health of the subject administered with the drug. The effect is produced in order to treat, prevent a disease, a defect or pathological condition or to alleviate some of the manifestations of a disease, defect or pathological condition.

The invention also encompasses chemical modifications of the compounds of formula I to prolong their circulating lifetimes. Examples of suitable poly(ethylene glycol) derivatives that possess this property are described in e.g. US 2005171328 (NEKTAR THERAPEUTICS AL CORP) or U.S. Pat. No. 6,713,454 (NOBEX CORP). Since the compounds of formula I are fairly lipophilic, the PEG-oligomer/polymer also increases the hydrophilicity of the pro-drugs and thereby their aqueous solubility.

The term “lipophilic.” should be understood as a molecular fragment that provides a lipophilic character to the full molecule to which it is attached, the lipophilic character being understood as a property that procures higher penetrability through biological membranes. Examples of such fragments include, but are not limited to, alkyl and alkenyl chains, aromatic groups, mono- or poly-fluorinated. Preferably, the full molecule resulting from attachment through such a lipophilic group will have CLogP values higher than 2. The compounds of the present invention will have preferably CLogP values higher than 3.

The selection method and the process method of an appropriate prodrug derivative are described in the literature such as Design of Prodrugs, Elsevier, Amsterdam 1985; G. R. Pettit et al. Anti-Cancer Drug Design 16 (2001) 185-193.

The compound(s) of formula (I) according to the present invention, their pharmaceutically acceptable salts or solvates or pro-drugs thereof, where applicable, may be administered in the form of a pharmaceutical composition in which they are in association with a pharmaceutically acceptable adjuvant, diluent or carrier, in order to prevent or treat any disease in which the compounds of the present invention would be considered beneficial by the skilled person. Preferably said diseases are cancers presenting tumor initiating cells. More preferably said diseases are cancers presenting glioma-initiating cells.

The present invention also provides a pharmaceutical composition comprising a compound of the invention, or a pharmaceutically acceptable salt or solvate thereof, as hereinbefore defined, in association with a pharmaceutically acceptable adjuvant, diluent or carrier. As to the appropriate excipients, diluents, adjuvants and carriers, reference may be made to the standard literature describing these, e.g. to chapter 25.2 of Vol. 5 of “Comprehensive Medicinal Chemistry”, Pergamon Press 1990, and to “Lexikon der Hilfsstoffe für Pharmazie, Kosmetik und angrenzende Gebiete”, by H. P. Fiedler, Editio Cantor, 2002. The term “pharmaceutically acceptable carrier or excipient” means a carrier or excipient that is useful in preparing a pharmaceutical composition that is generally safe, and possesses acceptable toxicities. Acceptable carriers or excipients include those that are acceptable for veterinary use as well as human pharmaceutical use. A “pharmaceutically acceptable carrier or excipient” as used in the specification and claims includes both one and more than one such carrier or excipient.

Optionally, the pharmaceutical composition of the present invention further comprises one or more additional active agents.

The compounds of the invention that are used in the methods of the present invention can be incorporated into a variety of formulations and medicaments for therapeutic administration. More particularly, a compound as provided herein can be formulated into pharmaceutical compositions by combination with appropriate, pharmaceutically acceptable carriers or diluents, and can be formulated into preparations in solid, semi-solid, liquid or gaseous forms, such as tablets, capsules, pills, powders, granules, dragees, gels, slurries, ointments, solutions, suppositories, injections, inhalants and aerosols. As such, administration of the compounds can be achieved in various ways, including oral, buccal, rectal, parenteral, intraperitoneal, intradermal, transdermal, intracranial and/or intratracheal administration. Moreover, the compound can be administered in a local rather than systemic manner, in a depot or sustained release formulation. The compounds can be formulated with common excipients, diluents or carriers, and compressed into tablets, or formulated as elixirs or solutions for convenient oral administration, or administered by the intramuscular or intravenous routes. The compounds can be administered transdermally, and can be formulated as sustained release dosage forms and the like. The compounds can be administered alone, in combination with each other, or they can be used in combination with other known compounds.

Suitable formulations for use in the present invention are found in Remington's Pharmaceutical Sciences (Mack Publishing Company (1985) Philadelphia, Pa., 17th ed.), which is incorporated herein by reference. Moreover, for a brief review of methods for drug delivery, see, Lancer, Science (1990) 249:1527-1533, which is incorporated herein by reference.

Sustained-release preparations may be prepared. Suitable examples of sustained-release preparations include semi permeable matrices of solid hydrophobic polymers containing the compound of the invention, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and [gamma] ethyl-L-glutamate, non-degradable ethylene-vinyl acetate, degradable lactic acid-glycolic glycolic acid copolymers such as the LUPRON DEPOT™ (injectable microspheres composed of lactic acid-glycolic acid copolymer and leuprolide acetate), and poly-D-(−)-3-hydroxybutyric acid.

The compound of the present invention may also be entrapped in microcapsules prepared, for example, by coacervation techniques or by interfacial polymerization, for example, hydroxymethylcellulose or gelatin-microcapsules and poly(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed, (1980).

The pharmaceutical compositions described herein can be manufactured in a manner that is known to those of skill in the art, i.e., by means of conventional mixing, dissolving, granulating, dragee-making, levigating, emulsifying, encapsulating, entrapping or lyophilizing processes. The following methods and excipients are merely exemplary and are in no way limiting. For injection, the compound (and optionally another active agent) can be formulated into preparations by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. Preferably, the compounds of the present invention can be formulated in aqueous solutions, preferably in physiologically compatible buffers such as Hanks' solution. Ringer's solution, or physiological saline buffer. For transmucosal administration, penetrants appropriate to the barrier to be permeated are used in the formulation. Such penetrants are generally known in the art.

Preferably pharmaceutical formulations for parenteral administration include aqueous solutions of the active compounds in water-soluble form. Additionally, suspensions of the active compounds can be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances that increase the viscosity of the suspension, such as sodium carboxymethyl cellulose, sorbitol, or dextran. Optionally, the suspension can also contain suitable stabilizers or agents that increase the solubility of the compounds to allow for the preparation of highly concentrated solutions, Alternatively, the active ingredient can be in powder form for constitution with a suitable vehicle, e.g., sterile pyrogen-free water, before use.

The amount of a compound as provided herein that can be combined with a carrier material to produce a single dosage form will vary depending upon the disease treated, the mammalian species, and the particular mode of administration. However, as a general guide, suitable unit doses for the compounds of the present invention can, for example, preferably contain between 0.1 rag to about 1000 mg, between 1 mg to about 500 mg, and between 1 mg to about 300 mg of the active compound. In another example, the unit dose is between 1 mg to about 100 mg. Such unit doses can be administered more than once a day, for example, 2, 3, 4, 5 or 6 times a day, but preferably 1 or 2 times per day, so that the total dosage for a 70 kg adult is in the range of 0.001 to about 15 mg per kg weight of subject per administration. A preferred dosage is 0.01 to about 1.5 mg per kg weight of subject per administration, and such therapy can extend for a number of weeks or months, and in some cases, years. It will be understood, however, that the specific dose level for any particular patient will depend on a variety of factors including the activity of the specific compound employed; the age, body weight, general health, sex and diet of the individual being treated; the time and route of administration; the rate of excretion; other drugs that have previously been administered; and the severity of the particular disease undergoing therapy, as is well understood by those of skill in the area. A typical dosage can be one 1 mg to about 100 mg tablet or 1 mg to about 300 mg taken once a day, or, multiple times per day, or one time-release capsule or tablet taken once a day and containing a proportionally higher content of active ingredient. The time-release effect can be obtained by capsule materials that dissolve at different pH values, by capsules that release slowly by osmotic pressure, or by any other known means of controlled release. It can be necessary to use dosages outside these ranges in some cases as will be apparent to those skilled in the art.

Applicants have found that the compounds of the present invention are inhibitors of the activity of Complex (III) of the mitochondrial electron transport chain and are useful in methods of treatment and/or prevention of a subject, preferably a mammalian subject, more preferably a human subject, who is suffering from cancers presenting tumor-initiating cells (GICs), preferably cancers presenting glioma-initiating cells. Preferably cancers presenting tumor-initiating cells are selected from the group comprising human gliomas, schwanommas, metastasis to the brain, meningiomas, ependymomas, a metastatic cancer such as for example melanoma, breast cancer, colon cancer or lung cancer.

The present invention further provides a compound of formula I

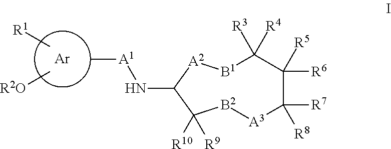

or a pharmaceutically acceptable salt or solvate thereof, wherein

Ar is selected from (C5-C10)aromatic ring or (C5-C10)heteroaromatic ring where one or more of the carbon, atoms in the ring system are replaced by heteroatoms selected from, the group consisting of O, S, and N;

R¹ is selected from H, —(C1-C10)alkyl, -aryl, —OH, —O—(C1-10)alkyl, —O-aryl, —NH₂, —NH(CHO), —NH(C═O)—(C1-10)alkyl, halogen, —NO₂, —C(═O)OH, —C(═O)O(C1-10)alkyl, —CF₃, —NH(C═O) (CF₃(OMe))Ph;

R² is selected from H, —(C1-10)alkyl, aryl, benzyl, —(CO) (CF₃(OMe))Ph;

R³ is selected from —H, —(C1-10)alkyl, —CF³ or can form a bond with R⁵;

R⁴, R⁹ and R¹⁰ are independently of each other selected from —H, —(C1-10)alkyl, —CF₃;

A¹, A² and A³ are independently of each other selected from —(C═O)—, —(C═S)—, —(S═O)—, —(S(═O)₂)—, —(CH₂)—, —(C(CH₃)₂)—, —CH(CF₃)—, —CHF—, —(C(CH₂OCH₂))—, —(C(CH₂SCH₂))—, —N(R^{a})—, —(CR′R″)—, —CH₂—CH₂—, —(C(CH₃)₂)—, where R^{a} is selected from —H, —(C1-C10)alkyl, —(C1-C10)alkenyl, —(C1-C10)alkynyl, mono or polyfluorinated (C1-C10)alkyl, —(C3-C10)cycloalkyl, —(C3-C10)heterocycloalkyl, -aryl, —(C1-C10)alkylaryl, -(C1-C10)alkylheteroaryl, —(C1-C10)alkyl-heterocycloalkyl, and where R′ and R″ are independently of each other selected from —H, —(C1-C10)alkyl, -halogen, —CF₃;

B¹ and B²:
(i) are independently of each other selected from —O—, —S—, —NH—, —CH₂—, or

(ii) form together —CH—CH— producing a bicyclo(3,4,0)-nonane unit.

(iii) form together —C═C— producing a bicycle

(iv) form together a —N—N— or —CH—N— fragment producing a diazabycycle or azabicycle

R⁵, R⁶, R⁷ and R⁸ are selected from:
(i) independently of each other, —R^{a}, —OR^{a}, —O(C═O)R^{a}, —C(═O)R^{a}, —C(═O)OR^{a}, —NR^{a}R^{b}, —NR^{a}(C═O)R^{b}, halogen, where R^{a} and R^{b} are independently of each other selected from —H, —(C1-C10)alkyl, —(C1-C10)alkenyl, —(C1-C10)alkynyl, mono or polyfluorinated (C1-C10)alkyl, —(C3-C10)cycloalkyl, —(C3-C10)heterocycloalkyl, -aryl, —(C1-C10)alkylaryl, —(C1-C10)alkylheteroaryl, —(C1-C10)alkyl-heterocycloalkyl or a combination thereof, or;
(ii) R⁵ and R⁷ form together a bond AND R⁶ and R⁸ are independently of each other selected from —R^{a}, —OR^{a}, —O(C═O)R^{a}, —C(═O)R^{a}, —C(═O)OR^{a}, —NR^{a}R^{b}, —NR^{a}(C═O)R^{b}, halogen, where R^{a} and R^{b} are independently of each other selected from —H, —(C1-C10)alkyl, —(C1-C10)alkenyl, —(C1-C10)alkynyl, mono or polyfluorinated (C1-C10)alkyl, —(C3-C10)cycloalkyl, —(C3-C10)heterocycloalkyl, -aryl, —(C1-C10)alkylaryl, —(C1-C10)alkylheteroaryl, —(C1-C10)alkyl-heterocycloalkyl or a combination thereof, or;
(iii) R⁵ and R⁷ represent independently of each other, —R^{a}, —OR^{a}, —O(C═O)R^{a}, —C(═O)R^{a}, —C(═O)OR^{a}, —NR^{a}Rb, —NR^{a}(C═O)R^{b}, halogen, where R^{a} and R^{b} represent independently of each other —H, —(C1-C10)alkyl, —(C1-C10)alkenyl, —(C1-C10)alkynyl, mono or polyfluorinated (C1-C10)alkyl, -aryl AND R⁶ and R⁸ form together a (C3-C10)cycloalkyl or (C3-C10)heterocycloalkyl substituted by R^{c} and R^{d} where R^{c} and R^{d} represent independently of each other —H, —(C1-C10)alkyl, —(C1-C10)alkenyl, —(C1-C10)alkynyl, mono or polyfluorinated (C1-C10)alkyl, -aryl, —OH, —O—(C1-C10)alkyl, —O—aryl, —O(C═O)—(C1-C10 )alkyl, —O(C═O)-aryl, halogen, or;
(iv) R⁵ and R⁷ form together a bond AND R⁶ and R⁸ form together a (C3-C10)cycloalkyl or heterocycloalkyl substituted by R^{c} and R^{d} where R^{c} and R^{d} represent independently of each other —H, —(C1-C10)alkyl, —(C1-C10)alkenyl, —(C1-C10)alkynyl, mono or polyfluorinated (C1-C10)alkyl, -aryl, —OH, —O—(C1-C10)alkyl, —O—aryl, —O(C═O)—(C1-C10)alkyl, —O(C═O)aryl, halogen, or;
(v) R⁵ and R⁷ form together a bond AND R⁶ and R⁸ form together an (C6-C10)aryl or (C5-C10)heteroaryl substituted by R^{c} and R^{d} where R^{c} and R^{d} represent independently of each other —H, —(C1-C10)alkyl, —(C1-C10)alkenyl, —(C1-C10)alkynyl, mono or polyfluorinated (C1-C10)alkyl, -aryl, —OH, —O—(C1-C10)alkyl, —O—aryl, —O(C═O)—(C1-C10)alkyl, —O(C═O)-aryl, halogen, or;
(vi) when B¹ and B² form together —C═C—, R³ and R⁵ form together a bona AND A³ is —C(R′R″)— or —N(R′)— AND R⁷ and R′ form together a bond, where R′ and R″ are independently of each other selected from —H, —(C1-C10)alkyl, -halogen, —CF₃;
   with the provision that Antimycin A is excluded, for use in a method for treating and/or preventing cancers presenting tumor-initiating cells.

Antimycin A is a chemical compound produced by Streptomyces bacteria. It is usually a mixture of several Antimycins A.

Preferably the compound of the present invention is for use in a method for treating and/or preventing cancers presenting tumor-initiating cells in a subject who has undergone a prior removal of a tumor/cancer bulk.

Preferably cancers presenting tumor-initiating cells are selected from the group comprising human gliomas, schwanommas, metastasis to the brain, meningiomas, ependymomas, a metastatic cancer.

Preferably a metastatic cancer is selected from the group comprising melanoma, breast cancer, colon cancer or lung cancer.

Preferably Ar is selected from (C5-C6) aromatic ring or (C5-C6) heteroaromatic ring where one or more of the carbon atoms in the ring system are replaced by heteroatoms selected from the group consisting of O, S, and N. More preferably Ar is (C5-C6)aromatic ring.

According to a preferred embodiment of the present invention, the compound of the invention of formula VI

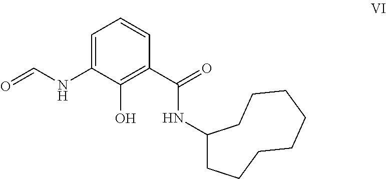 [SMILES: O=CNc1cccc(C(=O)NC2CCCCCCCC2)c1O]

is for use in a method for treating and/or preventing cancers presenting tumor-initiating cells.

More preferably the cancers presenting tumor-initiating cells is a cancer involving glioma-initiating cells (GICs).

In a further embodiment of the present invention, cancers presenting tumor initiating cells are recurrent tumors/cancers. The term “recurrent cancer” or “recurrent tumor”, refers to a cancer, for example glioma, that has recurred (come back), usually after a period of time during which the cancer could not be detected. The cancer may come back to the same place as the original (primary) tumor or to another place in the body of a subject.

In a particular embodiment, the present invention provides a method for treating or preventing cancers presenting tumor-initiating cells comprising administering to a subject in need of such treatment an effective amount of the compound of the invention or the pharmaceutical composition of the invention.

The daily dose of compounds of the present invention will necessarily be varied depending upon the host treated, the particular route of administration, and the severity and kind of the illness being treated. Accordingly the optimum dosage may be determined by the practitioner who is treating any particular patient. Further, it is noted that the clinician or treating physician will know how and when to start, interrupt, adjust, or terminate therapy in conjunction with individual patient response.

For any compound used in the method of the present invention, a therapeutically effective dose can be estimated initially from cell culture assays, animal models, or microdosing of human subjects.

“Treatment” refers to both therapeutic treatment and prophylactic or preventative measures. Those in need of treatment include those already with the disorder, for example cancer, as well as those in which the disorder, for example cancer, is to be prevented. Hence, the mammal, preferably human, to be treated herein may have been diagnosed as having the disorder, for example cancer, or may be predisposed or susceptible to the disorder, for example cancer.

“Mammal” for purposes of treatment refers to any animal classified as a mammal, including humans, domestic and farm animals or pet animals, such as dogs, horses, cats, cows, monkeys etc. Preferably, the mammal is human.

The term “therapeutically effective amount” refers to an amount of a drug effective to treat a disease or disorder in a mammal. In the case of cancer presenting tumor-initiating cells, the therapeutically effective amount of the drug may reduce the number of tumor-initiating cell, preferably glioma-initiating cells; reduce the tumor size; inhibit (i.e., slow to some extent and preferably stop) cancer cell and/or tumor-initiating cells infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the compounds of the present invention may prevent growth and/or kill existing cancer cells and/or tumor-initiating cells, it may be cytostatic and/or cytotoxic. The phrase “therapeutically effective amount” is used herein to mean an amount sufficient to prevent, or preferably reduce by at least about 30 percent, preferably by at least 50 percent, preferably by at least 70 percent, preferably by at least 80 percent, preferably by at least 90%, a clinically significant change in the growth or progression or mitotic activity of a target cellular mass, group of cancer cells or tumor-initiating cells, or other feature of pathology.

The terms “cancer” and “cancerous” refer to or describe the physiological condition in mammals that is typically characterized by unregulated cell growth. According to the present invention, cancer refers preferably to cancers presenting tumor-initiating cells (TICs), in particular human gliomas (GICs), schwanommas, metastasis to the brain, meningiomas, ependymomas, a metastatic cancer such as for example melanoma, breast cancer, colon cancer or lung cancer.

Optionally the compounds of the present invention may be used against cell proliferate diseases in combination (for example either at the same time, or almost, at the same time, or one after the other) with conventional treatments such as standard radiotherapy and/or standard chemotherapy. The standard radiotherapy and chemotherapy can be also the concomitant chemo-radiotherapy.

Therefore, optionally, the standard radiotherapy and/or chemotherapy can be performed before, simultaneously or after the administration of a therapeutically effective amount of the compound of the present invention, or pharmaceutical compositions containing thereof.

The term “concomitant chemo-radiotherapy” is used when these two treatments (chemotherapy and radiotherapy) are given either at the same time, or almost at the same time, for instance one after the other, or on the same day, etc.

The term “standard radiotherapy” refers to the use of ionizing radiation as part of cancer treatment to control malignant cells. Preferably the ionizing radiation is γ-irradiation. It is also common to combine radiotherapy with surgery, chemotherapy, hormone therapy, or combinations thereof. Most common cancer types can be usually treated with radiotherapy. The precise treatment intent (curative, adjuvant, neoadjuvant or palliative) will depend on the tumor type, location, and stage, as well as the general health of the subject in need thereof.

The term “standard chemotherapy” generally refers to a treatment of a cancer using specific chemotherapeutic/chemical agents. A chemotherapeutic agent refers to a pharmaceutical agent generally used for treating cancer. The chemotherapeutic agents for treating cancer include, for example, Altretamine, Bleomycin, Busulphan, Capecitabine, Carboplatin, Carmustine, Chlorambucil, Cisplatin, Cladribine, Crisantaspase, Cyclophosphamid, Cytarabine, Dacarbazine, Daunorubicin, Doxorubicin, Epirubicin, Etoposide, Fludarabine, Fluorouracil, Gemcitabine, Idarubicin, Ifosfamide, Irinotecan, Lomustine, Melphalan, Mercaptopurine, Methotrexate, Mitomycin, Mitoxantrone, Oxaliplatin, Pentostatin, Procarbazine, Streptozocin, Taco, Temozolomide, Tioguanine/Thioguanine, Thiotepa, Topotecan, Treosulfan, Vinblastine, Vincristine, Vindesine or Vinorelbine.

When a chemotherapeutic agent is used in combination with the compounds of formula (I) according to the present invention, then this may be used in the form of a medicament containing a combination of these two agents, for simultaneous administration, or they may be used in the form of separate dosage forms, each containing one of the agents, and in the latter case the individual dosage forms may be used e.g. sequentially, i.e. one dosage form with the compound (I), followed by a dosage form containing the chemotherapeutic agent (or vice versa). This embodiment of two separate dosage forms may be conceived and provided in the form of a kit.

Also optionally the compounds of the present invention may be used against cell proliferate diseases, such as cancers presenting tumor-initiating cells, preferably cancers presenting giioma-initiating cells, in combination with conventional removal of a tumor bulk, by for example segmental resection (biopsy or gross resection).

The term “removal of a tumor bulk” refers to any removal, ablation or resection of a tumor bulk from a subject. The removal can be chemical, radiation or surgical. Preferably said removal is surgical, such as ablation or resection. Resection can be “segmental resection” (or segmentectomy), a surgical procedure to remove part of an organ or gland from a subject. It may also be used to remove a tumor and normal tissue around it.

Debulking agent may be also used to remove tumor bulk. The term “debulking agent” includes any molecule (e.g. chemical, biological) or any external/environmental agent (e.g. γ-irradiation) or traditional surgery that would allow killing cancer cells from the tumor bulk (e.g. FL1⁰ and FL1⁻ cells as mentioned above).

According to an embodiment of the present invention, it may be advantageous that the compound of the present invention is used in a method for treating or preventing cancers presenting tumor-initiating cells in a subject who has undergone a prior removal of a tumor/cancer bulk in order to better target tumor-initiating cells.

Another object of the present invention is a kit comprising the compound of the present invention or the pharmaceutical composition of the present invention for use in a method for treatment and/or prevention of cancers presenting tumor-initiating cells.

Generally, the Kit comprises a container and a label or package insert on or associated with the container. Suitable containers include, for example, bottles, vials, syringes, etc. The containers may be formed from a variety of materials such as glass or plastic. The container holds the compound's composition or the pro-drug composition or pharmaceutically acceptable salts thereof that are effective for treating the condition and may have a sterile access port (for example the container may be an intravenous solution bag or a vial having a stopper pierceable by a hypodermic injection needle). The label or package insert indicates that the composition is used for treating the condition of choice, such as cancer.

Since the compounds of the present invention are inhibitors of the activity of Complex (III) of the mitochondrial electron transport chain, the present invention further provides the use of the compound of the invention for many other applications where the inhibition of the activity of Complex (III) of the mitochondrial electron transport chain is beneficial. This includes, for example, antibacterial, antifungal, pesticide and herbicide applications. Therefore the compounds of the present invention can be used in agriculture, in fish farming and in food industry in general.

In particular, the present invention provides for the use of the compound of the present invention as antibacterial agent, antifungal agent, pesticide agent and/or herbicide agent.

Another useful application of the compound(s) of the present invention is its use as cardiovascular drugs.

Those skilled in the art will appreciate that the invention described herein is susceptible to variations and modifications other than those specifically described. It is to be understood that the invention includes all such variations and modifications without departing from the spirit or essential characteristics thereof. The invention also includes all of the steps, features, compositions and compounds referred to or indicated in this specification, individually or collectively, and any and all combinations or any two or more of said steps or features. The present disclosure is therefore to be considered as in all aspects illustrated and not restrictive, the scope of the invention being indicated by the appended Claims, and all changes which come within the meaning and range of equivalency are intended to be embraced therein.

Various references are cited throughout this Specification, each of which is incorporated herein by reference in its entirety.

The foregoing description will be more fully understood with reference to the following Examples. Such Examples, are, however, exemplary of methods of practicing the present invention and are not intended to limit the scope of the invention.

### EXAMPLES

### Synthesis of Compounds

Typically the compounds of the present invention can be synthesised by adapting the protocols reported by Hu et al, in Tetrahedron Lett., 2008, 49, 5132, or Wu et al, in J. Org. Chem. 2006, 72, 4296 or Chakraborty et al. in Tetrahedron Lett. 2007, 48, 1265. Other heteroatora containing rings (bis-lactara, bis-thiolactones etc.) can be accessed using the same synthetic approach. The cycloalkyl and heterocycloakyl rings formed by for example by R⁵ and R⁶ can be introduced before the closure of the 9-membered ring using methods known by the person skilled in the art, or after closure of the ring, using for example ring-closing methatesis reactions followed by hydrogenation. Metathesis can also be used for the cyclisation of the 9-membered central ring. Examples of ring-closing methasis to synthesize 9-membered ring can be found in Clark et al., Org. Lett. 2003, 5, 89.

For examples where the central core is an all-carbon bicycle or a heteroatom containing bicycle, a person skilled in the art will synthesize the corresponding central motif using reactions such as Diels-Alder reaction, before decorating the scaffold with the desired side-chains.

Alternatively, the amino group attached to the ring can be introduced by reductive amination of the corresponding preformed cyclic ketone.

### Synthesis of Compound According to Formula VI:

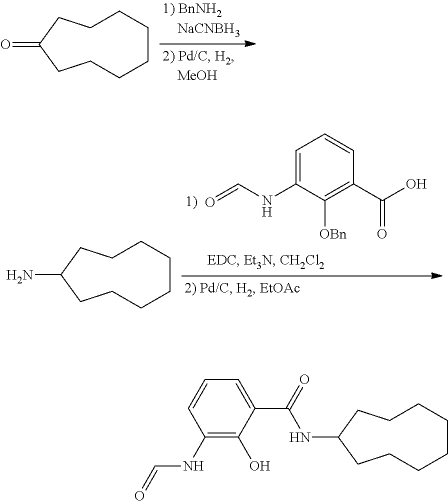 [SMILES: O=C1CCCCCCCC1 ; O=CNc1cccc(C(=O)O)c1OCc1ccccc1 ; NC1CCCCCCCC1 ; O=CNc1cccc(C(=O)NC2CCCCCCCC2)c1O]

Compound of formula VI was synthesized in four steps from cyclononanone. Reductive amination of cyclononanone using benzylamine and NaCNBH₃ afforded a secondary benzyl amine that was purified by flash chromatography (SiO₂, CH₂Cl₂/MeOH) to afford colourless oil. Treatment of this oil under hydrogenolytic conditions (Pd/C, 1 atm H₂, MeOH) followed by filtration and concentration afforded a crude primary amine that was used without purification. Coupling between this amine and the known amino-salicylic acid derivative afforded an amide that was treated with Pd and H₂ (1 atm) in EtOAc. Upon filtration, concentration and chromatographic purification (SiO₂, CH₂Cl₂/MeOH), the desired compound of formula VI was obtained as a off-white solid.

### Example 1

### 3-formamido-2-hydroxy-N-(4-methyl-2,6-dioxo-1,5-dioxonan-3-yl)benzamide

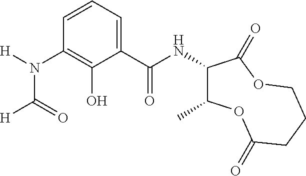 [SMILES: C[C@H]1OC(=O)CCCOC(=O)[C@H]1NC(=O)c1cccc(NC=O)c1O]

¹H NMR (400 MHz, CDCl3): δ 12.65 and 12.50 (2×s, total integr. 1H), 8.78 and 8.50 (d, J=11.4 Hz, and d, J=1.3 Hz, total integr, 1H), 8.55 and 7.40 (dd, J=8.0, 0.9 Hz, and br s, total integr. 1H), 7.98 and 7.78 (br s and br d, J=11.6 Hz, total integr. 1H), 7.30 and 7.25 (br d, J=7.6 Hz, and dd, J=8.0, 1.2 Hz, total integr. 1H), 7.15 (br d, J=7.2 Hz, 1H), 6.92 (t, J=3.0 Hz, 1H), 5.70-5.62 (m, 1H), 5.21 (t, J=7.2 Hz, 1H), 4.93 (ddd, J=10.8, 7.0, 3.7 Hz, 1H), 4.11 (ddd, J=11.2, 7.6, 3.8 Hz, 1H), 2.50-2.41 (m, 2H), 2.30-2.20 (m, 1H), 2.06-1.96 (m, 1H), 1.33 (d, J=6.7 Hz, 3H); MS (ES⁺): m/z: Calcd for C₁₆H₁₈N₂O₇ Na (M⁺+Na): 373.1, found: 373.1.

### Example 2

### N-(2,3-dihydro-1H-inden-2-yl)-3-formamido-2-hydxroxybenzamide

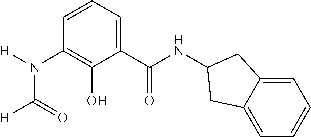 [SMILES: O=CNc1cccc(C(=O)NC2Cc3ccccc3C2)c1O]

¹H NMR (400 MHz, CDCl₃): δ13.28 and 13.99 (2×s, total integr. 1H), 8.77 and 8.48 (d, J=11.5 Hz, and d, J=1.7 Hz, total integr. 1H), 8.50 (dd, J=8.0, 1.3 Hz, 1H), 7.93 and 7.77 (br s and br d, J=11.0 Hz, total integr, 1H), 7.31-7.18 (m, 4H), 7.07 and 7.02 (dd, J=8.1, 1.4 Hz, and dd, J=8.3, 1.1 Hz, total integr. 1H), 6.81 (t, J=8.1 Hz, 1H), 6.54 (br d, J=8.5 Hz, 1H), 4.98-4.88 (m, 1H), 3.44 (dd, J=16.4, 7.4 Hz, 2H), 2.95 (dd, J=16.4, 3.9 Hz, 2H); HRMS (ES⁺): m/z: Calcd for C₁₇H₁₆N₂O₃ (M+H⁺): 297.1228, found: 297.1234.

### Example 3

### N-Cyclooctyl-3-formamido-2-hydroxybenzamide

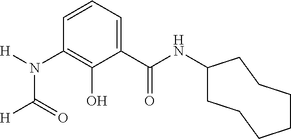 [SMILES: O=CNc1cccc(C(=O)NC2CCCCCCC2)c1O]

¹H NMR (400 MHz, CDCl₃): δ 13.31 and 13.12 (2×s, total integr. 1H), 8.78 and 8.49 (d, J=10.8 Hz, and d, J=1.8 Hz, total integr. 1H), 8.50 and 7.30 (dd, J=8.0, 1.3 Hz, and br d, J=8.5 Hz, total integr. 1H), 7.94 and 7.77 (br s and br d, J=11.5 Hz, total integr. 1H), 7.12 and 7.08 (br d, J=8.0 Hz, and dd, J=7.7, 1.8 Hz, total integr. 1H), 6.85 (t, J=8.1 Hz, 1H), 6.27 (br d, J=8.0 Hz, 1H), 4.24-4.15 (m, 1H), 1.98-1.88 (m, 2H), 1.76-1.54 (m, 12H); MS (ES⁺): m/z: Calcd for C₁₆H₂₂N₂O₃ Na (M⁺+Na); 313.1, found: 313.1.

### Example 4

### N-cyclononyl-3-formamido-2-hydroxybenzamide

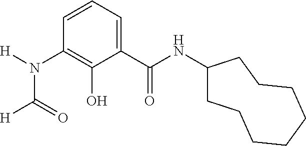 [SMILES: O=CNc1cccc(C(=O)NC2CCCCCCCC2)c1O]

¹H NMR (400 MHz, CDCl₃): δ 13.31 and 13.12 (2×s, total integr. 1H), 8.78 and 8.49 (d, J=11.7 Hz, and d, J=1.6 Hz, total integr. 1H), 8.50 and 7.30 (dd, J=8.0, 1.4 Hz, and br d, J=8.5 Hz, total integr, 1H), 7.94 and 7.77 (br s and br d, J=12.2 Hz, total integr. 1H), 7.12 and 7.07 (br d, J=7.7 Hz, and dd, J=8.0, 1.4 Hz, total integr. 1H), 6.85 (t, J=8.0 Hz, 1H), 6.25 (br d, J=7.5 Hz, 1H), 4.31-4.22 (m, 1H), 1.76-1.54 (m, 16H); HRMS (ES⁺): m/z: Calcd for C₁₇H₂₄N₂O₃ (M+H⁺): 305.1860, found: 305.1866.

### Example 5

### (R)-3-formamido-2-hydroxy-N-(2-oxocyclooctcyl)benzeamide

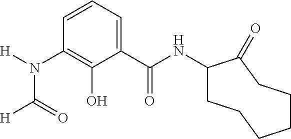 [SMILES: O=CNc1cccc(C(=O)NC2CCCCCCC2=O)c1O]

¹H NMR (400 MHz, CDCl₃): δ 12.99 and 12.81 (2×s, total integr. 1H), 8.78 and 8.49 (d, J=11.9 Hz, and d, J=1.7 Hz, total integr. 1H), 8.50 and 7.33 (dd, J=10.4, 1.3 Hz, and br d, J=8.2 Hz, total integr. 1H), 7.93 and 7.78 (br s and br d, J=12.2 Hz, total integr. 1H), 7.39 (br d, J=4.7 Hz, 1H), 7.30 and 7.26 (br d, J=8.0 Hz, and dd, J=8.0, 1.3 Hz, total integr. 1H), 6.89 (t, J=8.0 Hz, 1H), 4.79-4.74 (m, 1H), 2.80 (ddd, J=13.5, 11.1, 3.4 Hz, 1H), 2.49-2.37 (m, 2H), 2.22-2.03 (m, 2H), 1.93-1.41 (m, 8H); HRMS (ES⁺): m/z: Calcd for C₁₆H₂₀N₂O₄ (M+H⁺): 305.1501, found; 305.1496.

### Example 6

### 3-formamido-2-hydroxy (2-oxocyclononyl)benamide

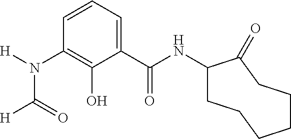 [SMILES: O=CNc1cccc(C(=O)NC2CCCCCCC2=O)c1O]

¹H NMR (400 MHz, CDCl₃): δ 13.00 and 12.83 (2×s, total integr. 1H), 8.78 and 8.49 (d, J=11.7 Hz, and d, J=1.4 Hz, total integr. 1H), 8.50 and 7.33 (dd, J=8.2, 1.3 Hz, and br d, J=7.6 Hz, total integr, 1H), 7.92 and 7.76 (br s and br d, J=12.2 Hz, total integr. 1H), 7.53 (br d, J=5.6 Hz, 1H), 7.30 and 7.26 (br d, J=8.6 Hz, and dd, J=8.2, 1.2 Hz, total integr. 1H), 6.90 (t, J=8.2 Hz, 1H), 4.75-4.70 (m, 1H), 2.86 (ddd, J=14.3, 9.9, 4.20 Hz, 1H), 2.45-2.23 (m, 3H), 1.96-1.83 (m, 2H), 1.72-1.35 (m, 8H); HRMS (ES⁺): m/z: Calcd for C₁₇H₂₂N₂O₄ (M+H⁺): 319.1, found: 319.1.

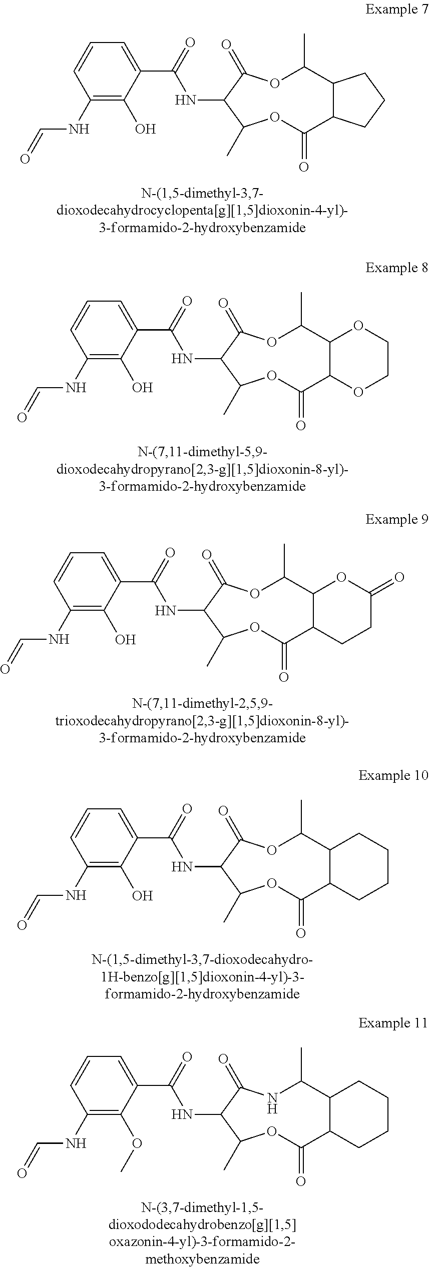 [SMILES: CC1OC(=O)C2CCCC2C(C)OC(=O)C1NC(=O)c1cccc(NC=O)c1O ; CC1OC(=O)C2OCCOC2C(C)OC(=O)C1NC(=O)c1cccc(NC=O)c1O ; CC1OC(=O)C2CCC(=O)OC2C(C)OC(=O)C1NC(=O)c1cccc(NC=O)c1O ; CC1OC(=O)C2CCCCC2C(C)OC(=O)C1NC(=O)c1cccc(NC=O)c1O ; COc1c(NC=O)cccc1C(=O)NC1C(=O)NC(C)C2CCCCC2C(=O)OC1C]

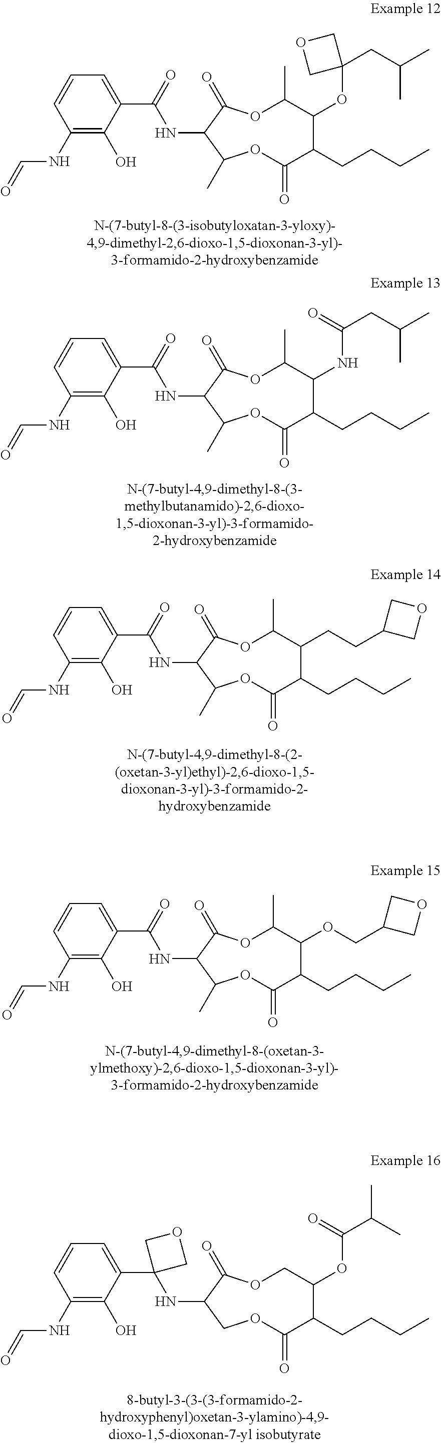 [SMILES: CCCCC1C(=O)OC(C)C(NC(=O)c2cccc(NC=O)c2O)C(=O)OC(C)C1OC1(CC(C)C)COC1 ; CCCCC1C(=O)OC(C)C(NC(=O)c2cccc(NC=O)c2O)C(=O)OC(C)C1NC(=O)CC(C)C ; CCCCC1C(=O)OC(C)C(NC(=O)c2cccc(NC=O)c2O)C(=O)OC(C)C1CCC1COC1 ; CCCCC1C(=O)OC(C)C(NC(=O)c2cccc(NC=O)c2O)C(=O)OC(C)C1OCC1COC1 ; CCCCC1C(=O)OCC(NC2(c3cccc(NC=O)c3O)COC2)C(=O)OCC1OC(=O)C(C)C]

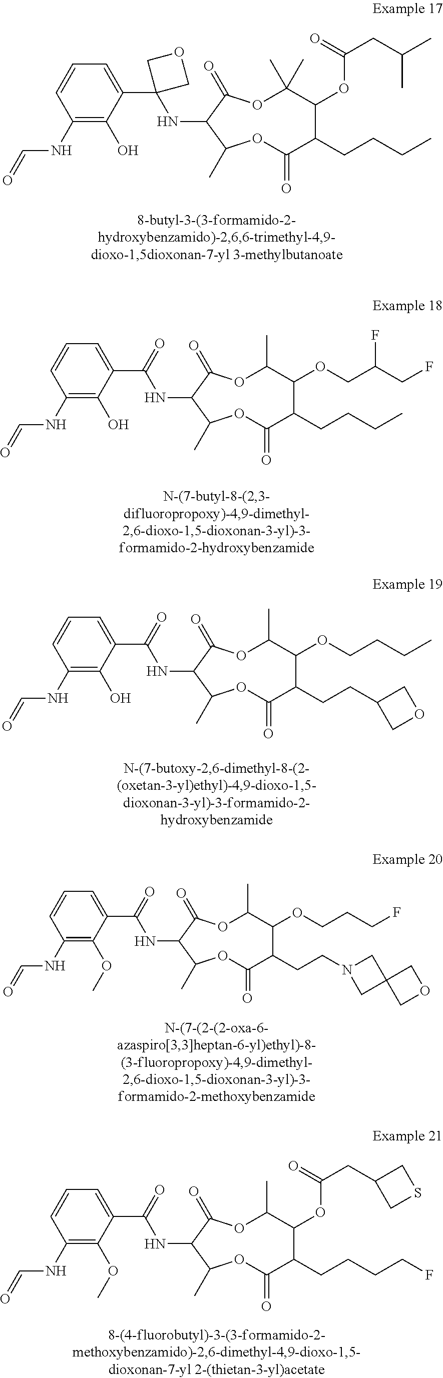 [SMILES: CCCCC1C(=O)OC(C)C(NC2(c3cccc(NC=O)c3O)COC2)C(=O)OC(C)(C)C1OC(=O)CC(C)C ; CCCCC1C(=O)OC(C)C(NC(=O)c2cccc(NC=O)c2O)C(=O)OC(C)C1OCC(F)CF ; CCCCOC1C(C)OC(=O)C(NC(=O)c2cccc(NC=O)c2O)C(C)OC(=O)C1CCC1COC1 ; COc1c(NC=O)cccc1C(=O)NC1C(=O)OC(C)C(OCCCF)C(CCN2CC3(COC3)C2)C(=O)OC1C ; COc1c(NC=O)cccc1C(=O)NC1C(=O)OC(C)C(OC(=O)CC2CSC2)C(CCCCF)C(=O)OC1C]

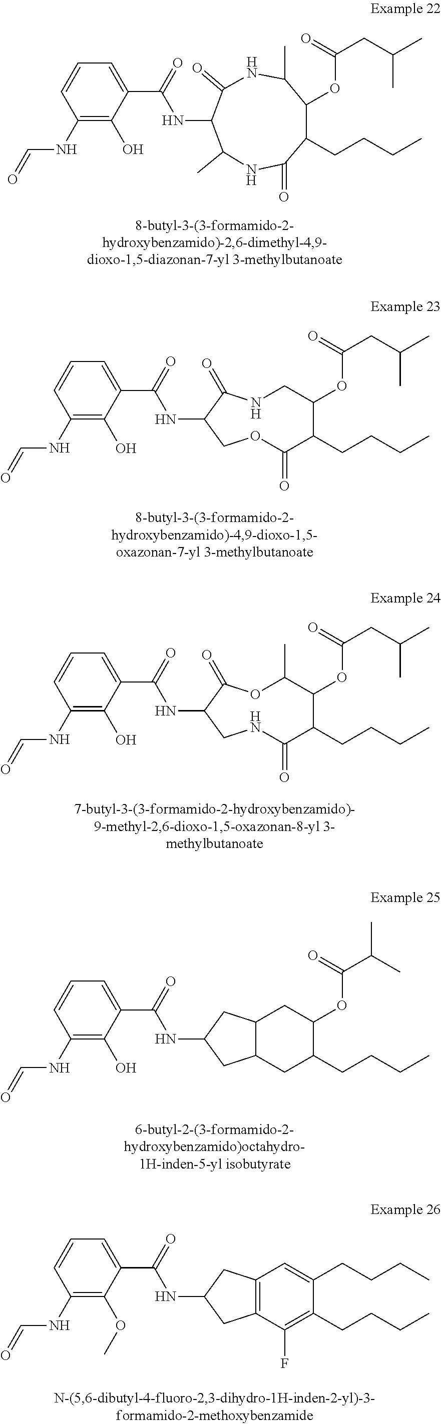 [SMILES: CCCCC1C(=O)NC(C)C(NC(=O)c2cccc(NC=O)c2O)C(=O)NC(C)C1OC(=O)CC(C)C ; CCCCC1C(=O)OCC(NC(=O)c2cccc(NC=O)c2O)C(=O)NCC1OC(=O)CC(C)C ; CCCCC1C(=O)NCC(NC(=O)c2cccc(NC=O)c2O)C(=O)OC(C)C1OC(=O)CC(C)C ; CCCCC1CC2CC(NC(=O)c3cccc(NC=O)c3O)CC2CC1OC(=O)C(C)C ; CCCCc1cc2c(c(F)c1CCCC)CC(NC(=O)c1cccc(NC=O)c1OC)C2]

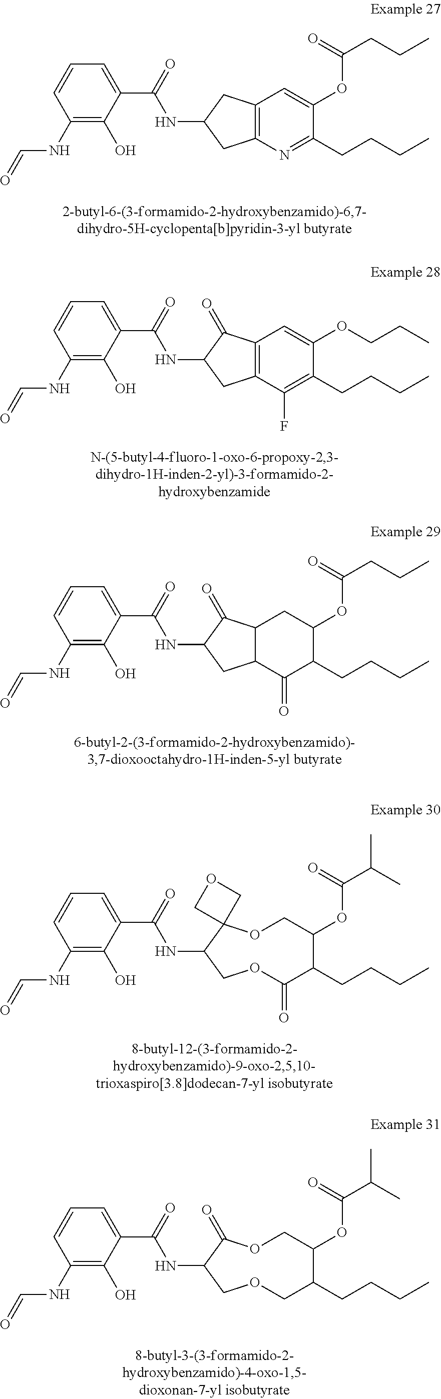 [SMILES: CCCCc1nc2c(cc1OC(=O)CCC)CC(NC(=O)c1cccc(NC=O)c1O)C2 ; CCCCc1c(OCCC)cc2c(c1F)CC(NC(=O)c1cccc(NC=O)c1O)C2=O ; CCCCC1C(=O)C2CC(NC(=O)c3cccc(NC=O)c3O)C(=O)C2CC1OC(=O)CCC ; CCCCC1C(=O)OCC(NC(=O)c2cccc(NC=O)c2O)C2(COC2)OCC1OC(=O)C(C)C ; CCCCC1COCC(NC(=O)c2cccc(NC=O)c2O)C(=O)OCC1OC(=O)C(C)C]

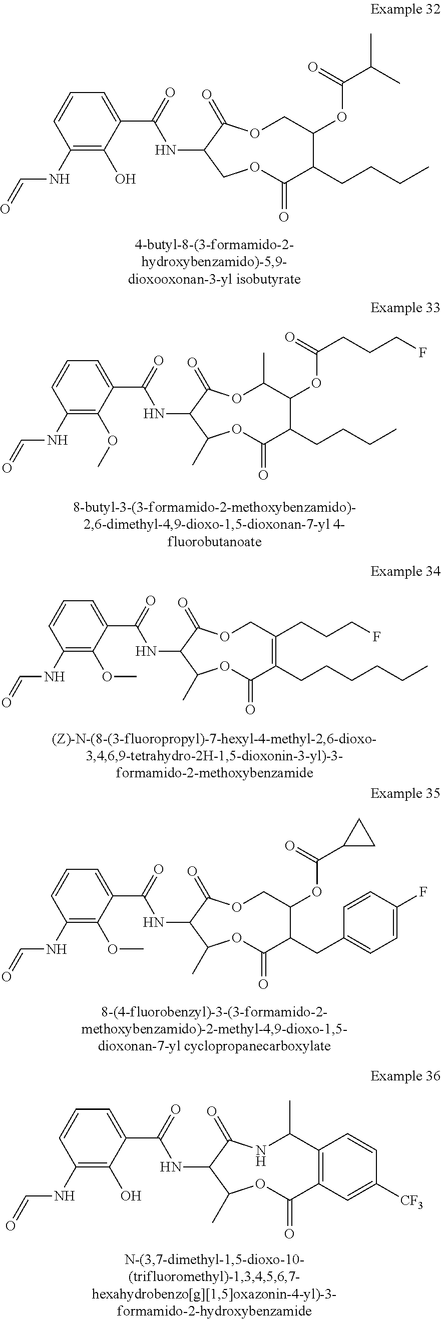 [SMILES: CCCCC1C(=O)OCC(NC(=O)c2cccc(NC=O)c2O)C(=O)OCC1OC(=O)C(C)C ; CCCCC1C(=O)OC(C)C(NC(=O)c2cccc(NC=O)c2OC)C(=O)OC(C)C1OC(=O)CCCF ; CCCCCC/C1=C(\CCCF)COC(=O)C(NC(=O)c2cccc(NC=O)c2OC)C(C)OC1=O ; COc1c(NC=O)cccc1C(=O)NC1C(=O)OCC(OC(=O)C2CC2)C(Cc2ccc(F)cc2)C(=O)OC1C ; CC1NC(=O)C(NC(=O)c2cccc(NC=O)c2O)C(C)OC(=O)c2cc(C(F)(F)F)ccc21]

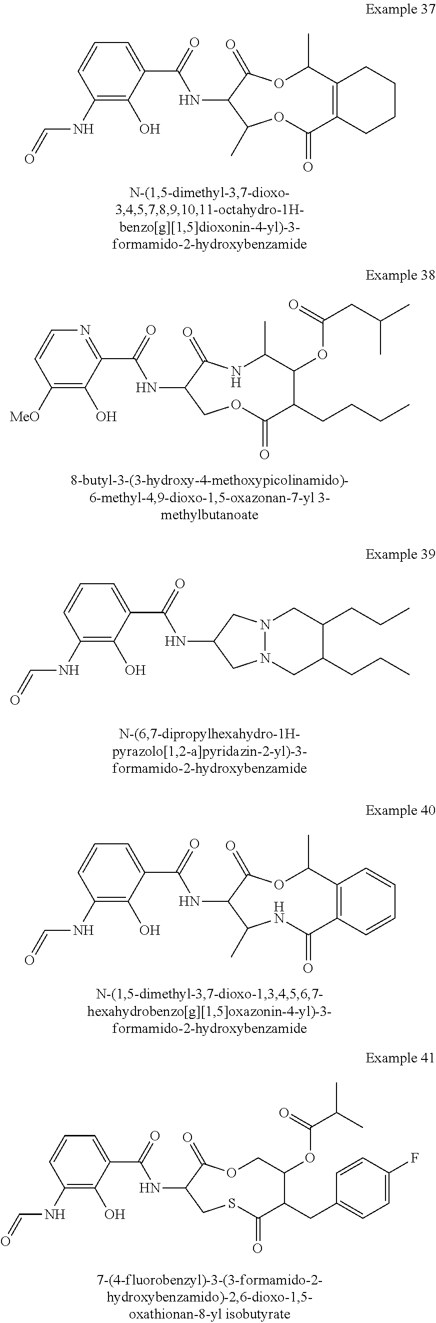 [SMILES: CC1OC(=O)C(NC(=O)c2cccc(NC=O)c2O)C(C)OC(=O)C2=C1CCCC2 ; CCCCC1C(=O)OCC(NC(=O)c2nccc(OC)c2O)C(=O)NC(C)C1OC(=O)CC(C)C ; CCCC1CN2CC(NC(=O)c3cccc(NC=O)c3O)CN2CC1CCC ; CC1OC(=O)C(NC(=O)c2cccc(NC=O)c2O)C(C)NC(=O)c2ccccc21 ; CC(C)C(=O)OC1COC(=O)C(NC(=O)c2cccc(NC=O)c2O)CSC(=O)C1Cc1ccc(F)cc1]

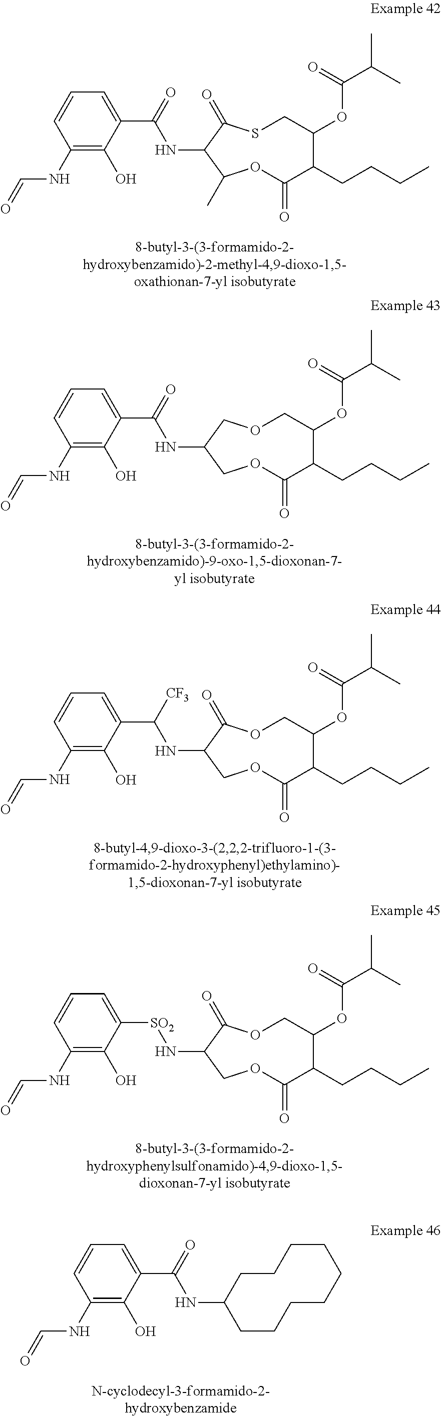 [SMILES: CCCCC1C(=O)OC(C)C(NC(=O)c2cccc(NC=O)c2O)C(=O)SCC1OC(=O)C(C)C ; CCCCC1C(=O)OCC(NC(=O)c2cccc(NC=O)c2O)COCC1OC(=O)C(C)C ; CCCCC1C(=O)OCC(NC(c2cccc(NC=O)c2O)C(F)(F)F)C(=O)OCC1OC(=O)C(C)C ; CCCCC1C(=O)OCC(NS(=O)(=O)c2cccc(NC=O)c2O)C(=O)OCC1OC(=O)C(C)C ; O=CNc1cccc(C(=O)NC2CCCCCCCCC2)c1O]

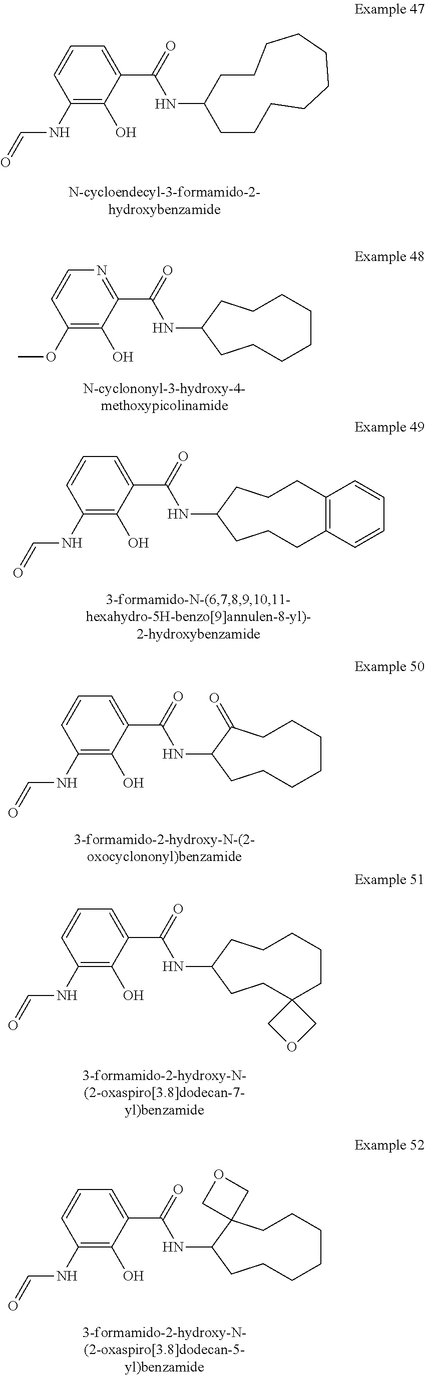 [SMILES: O=CNc1cccc(C(=O)NC2CCCCCCCCCC2)c1O ; COc1ccnc(C(=O)NC2CCCCCCCC2)c1O ; O=CNc1cccc(C(=O)NC2CCCc3ccccc3CCC2)c1O ; O=CNc1cccc(C(=O)NC2CCCCCCCC2=O)c1O ; O=CNc1cccc(C(=O)NC2CCCCCC3(CC2)COC3)c1O ; O=CNc1cccc(C(=O)NC2CCCCCCCC23COC3)c1O]

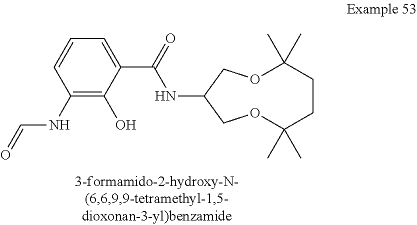 [SMILES: CC1(C)CCC(C)(C)OCC(NC(=O)c2cccc(NC=O)c2O)CO1]

### Determination of the Efficacy of Compounds

Short term dose response of the compound of Example 4 was performed according to the method as described in the patent application PCT no PCTIB2010/052237 (WO 2010/134039).

Briefly, gliomaspheres were dissociated, counted and plated at 10 cells/μl in DMEM-F12, 2% B27, 1% peni/strep, EGF and bFGF at 1 ng/ml. Cells were then treated with various amount of the compound for 48 hrs and cell death was analysed by FACS after incorporation of trypan blue (1/1000).

| Compound | Structure |
|---|---|
| Negative control | 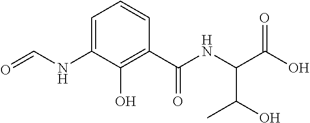 [SMILES: CC(O)C(NC(=O)c1cccc(NC=O)c1O)C(=O)O] |
| Compound of Example 4 | 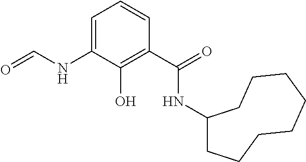 [SMILES: O=CNc1cccc(C(=O)NC2CCCCCCCC2)c1O] |

### Results

The treatment of glioma-initiating cells (GICs) with increasing doses of the compound of Example 4 decreases cell viability at 48 hrs, by inducing a fold increase≧1.2 cell death at 50 μM in contrast to the negative control compound.

These results suggest that the family of molecules derived from the compound of Example 4 might be considered as specific and efficient against tumor-initiating cells, preferably glioma-initiating cells.

Determination of the IC50 using MTT Assay

### Protocol Summary

Human hepatocellular liver carcinoma cells (HepG2) or Human glioblastoma-astrocytoma, epithelial-like cells (U-87 MG) were plated on 96-well tissue culture treated polystyrene plates at 0.5×10⁴ cells in 100 μL of Dulbecco's modified Eagle's medium containing either galactose or glucose supplemented with 10% foetal bovine serum and antibiotics per well. After 24 hr the cells are dosed with test compound at a range of concentrations and incubated for 72 nr. One hour prior to the end of the incubation period, the cells are loaded with MTT [yellow; 3-(4,5-dimethyl-2-thiazolyl)-2,5-diphenyl-2H-tetrazolium bromide], the plates are dried and re-solubilised using DMSO. The plates are then scanned using SpectraFluor Plus (TECAN).

### Objective

To measure the relative sensitivity of HepG2 or U-87 MG cells grown in medium containing either galactose or glucose to the test compound.

### Assay Sensitivity

Cytotoxicity was assessed using MTT. The assay provides a measurement of mitochondrial dehydrogenase activity and cell loss.

Cell Loss: A decrease can indicate a loss of cells indicating toxicity due to necrosis, apoptosis or a reduction in cellular proliferation.

Mitochondrial Activity: A decrease can also indicate an effect on mitochondrial function as mitochondrial dehydrogenases reduce the MTT [yellow; 3-(4,5-dimethyl-2-thiazolyl)-2,5-diphenyl-2H-tetrazolium bromide] to formazan. The formazan is detected in this assay (see assay protocol for details).

### Determining Mitochondrial Toxicity

The AC₅₀ is calculated from a four point logistic curve fit to determine percent survival of each test agent and control compound. The fold change is calculated from AC₅₀ curve fit data by comparison with cells grown with glucose or galactose, A greater than 5-fold response in the presence of galactose indicates the compound is considered to be a mitochondrial toxicant.

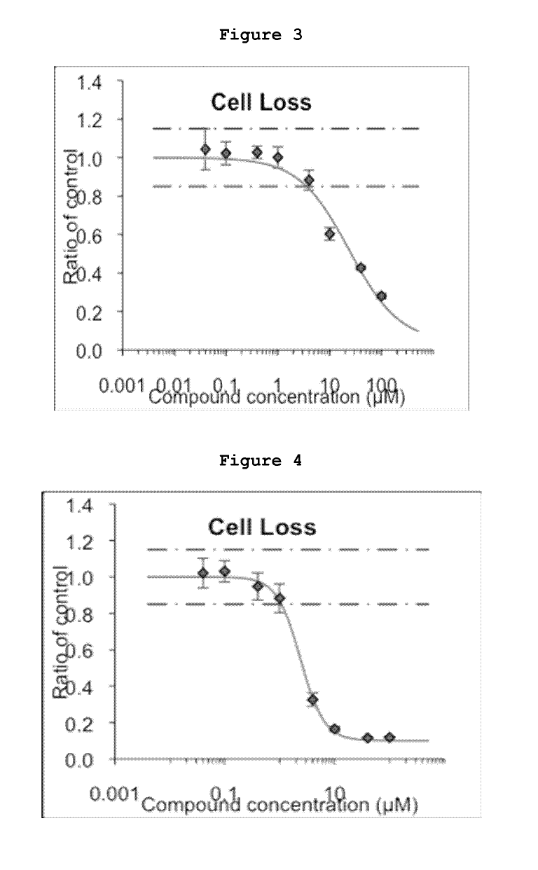

### Results for the Compound of Example 4 in HepG2 Cells

| Incubation Time: | 72 h | |
|---|---|---|
| Cell Line: | HepG2 | |
| Concentrations (μM): | 0.04, 0.1, 0.4, 1, 4, 10, 40, 100 | |
| Replicates per | 3 | |
| concentration: | | |
| Cell Health Parameter | MEC (μM) | AC50 (μM) |
| Cell Loss - Glucose | 10 | 23.8 |
| See FIG. 3 | | |
| Cell Loss - Galactose | 4 | 2.40 |
| See FIG. 4 | | |

| | | |
|---|---|---|
| Mitochondrial Toxicant | Yes | Fold Change = |
| | | 9.92 |

MEC = Minimum effective concentration that significantly crosses vehicle control threshold.
AC₅₀ = The concentration at which 50% maximum effect is observed for each cell health parameter.

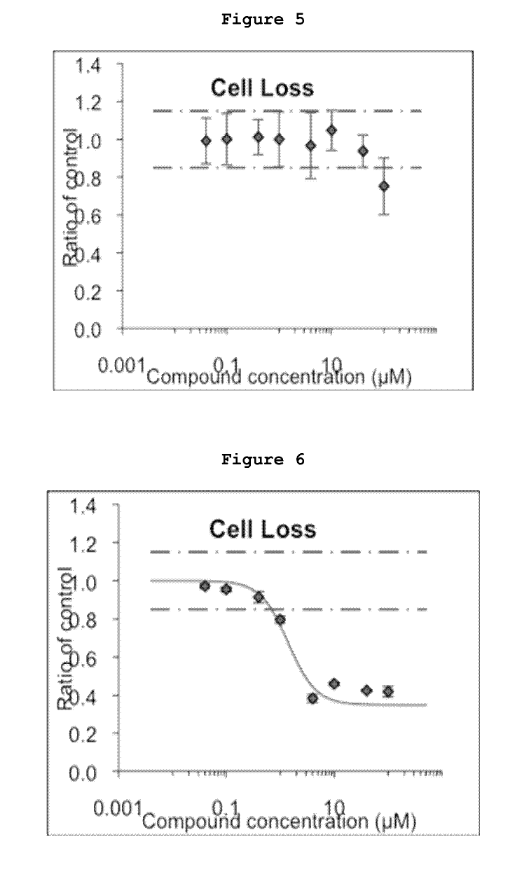

### Results for the Compound of Example 4 in U-87 MG Cells

| Incubation Time: | 72 h | |
|---|---|---|
| Cell Line: | U-87 MG | |
| Concentrations (μM): | 0.04, 0.1, 0.4, 1, 4, 10, 40, 100 | |
| Replicates per | 3 | |
| concentration: | | |
| Cell Health Parameter | MEC (μM) | AC50 (μM) |
| Cell Loss - Glucose | >100 | >100 |
| See FIG. 5 | | |
| Cell Loss - Galactose | 1 | 1.41 |
| See FIG. 6 | | |

| | | |
|---|---|---|
| Mitochondrial Toxicant | Yes | Fold Change = |
| | | >70.1 |

MEC = Minimum effective concentration that significantly crosses vehicle control threshold.
AC₅₀ = The concentration at which 50% maximum effect is observed for each cell health parameter.

1.-35. (canceled)

36. A compound of formula I 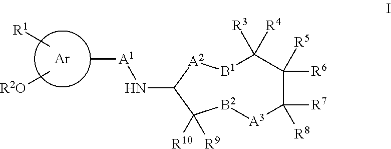
or a pharmaceutically acceptable salt or solvate thereof, wherein
Ar is selected from (C5-C10)aromatic ring or (C5-C10)heteroaromatic ring where one or more of the carbon atoms in the ring system are replaced by heteroatoms selected from the group consisting of O, S, and N;
R¹ is selected from H, —(C1-C10)alkyl, -aryl, —OH, —O—(C1-10)alkyl, —O—aryl, —NH₂, —NH(CHO), —NH(C═O)—(C1-10)alkyl, halogen, —NO₂, —C(═O)OH, —C(═O)O(C1-10)alkyl, —CF₃, and —NH(C═O)(CF₃(OMe))Ph;
R² is selected from H, —(C1-10)alkyl, aryl, benzyl, and —(C═O)(CF₃(OMe))Ph;
R³ is selected from —H, —(C1-10)alkyl, and —CF₃, or can form a bond with R⁵;
R⁴, R⁹ and R¹⁰ are independently of each other selected from —H, —(C1-10)alkyl, and —CF₃;
A¹, A² and A³ are independently of each other selected from —(C═O)—, —(C═S)—, —(S═O)—, —(S(═O)₂)—, —(CH₂)—, —(C(CH₃)₂)—, —CH(CF₃)—, —CHF—, —(C(CH₂OCH₂))—, —(C(CH₂SCH₂))—, —N(R^{a})—, —(CR′R″)—, —CH₂—CH₂—, and —(C(CH₃)₂)—, where R^{a} is selected from —H, —(C1-C10)alkyl, —(C1-C10)alkenyl, —(C1-C10)alkynyl, mono or polyfluorinated (C1-C10)alkyl, —(C3-C10)cycloalkyl, —(C3-C10)heterocycloalkyl, -aryl, —(C1-C10)alkylaryl, -(C1-C10)alkylheteroaryl, and —(C1-C10)alkyl-heterocycloalkyl, and where R′ and R″ are independently of each other selected from —H, —(C1-C10)alkyl, -halogen, and —CF₃;
B¹ and B²:
(i) are independently of each other selected from —O—, —S—, —NH—, and —CH₂—, or
(ii) form together —CH—CH— producing a bicyclo(3,4,0)-nonane unit;
(iii) form together —C═C— producing a bicycle;
(iv) form together a —N—N— or —CH—N— fragment producing a diazabycycle or azabicycle;
R⁵, R⁶, R⁷ and R⁸ are selected from:
(i) independently of each other, —R^{a}, —OR^{a}, —O(C═O)R^{a}, —C(═O)R^{a}, —C(═O)OR^{a}, —NR^{a}R^{b}, —NR^{a}(C═O)R^{b}, and halogen, where R^{a} and R^{b} are independently of each other selected from —H, —(C1-C10)alkyl, —(C1-C10)alkenyl, —(C1-C10)alkynyl, mono or polyfluorinated (C1-C10)alkyl, —(C3-C10)cycloalkyl, —(C3-C10)heterocycloalkyl, -aryl, —(C1-C10)alkylaryl, —(C1-C10)alkylheteroaryl, —(C1-C10)alkyl-heterocycloalkyl and a combination thereof, or;
(ii) R⁵ and R⁷ form together a bond and R⁶ and R⁸ are independently of each other selected from —R^{a}, —OR^{a}, —O(C═O)R^{a}, —C(═O)R^{a}, —C(═O)OR^{a}, —NR^{a}R^{b}, —NR^{a}(C═O)R^{b}, and halogen, where R^{a} and R^{b} are independently of each other selected from —H, —(C1-C10)alkyl, —(C1-C10)alkenyl, —(C1-C10)alkynyl, mono or polyfluorinated (C1-C10)alkyl, —(C3-C10)cycloalkyl, —(C3-C10)heterocycloalkyl, -aryl, —(C1-C10)alkylaryl, —(C1-C10)alkylheteroaryl, —(C1-C10)alkyl-heterocycloalkyl and a combination thereof, or;
(iii) R⁵ and R⁷ represent independently of each other, —R^{a}, —OR^{a}, —O(C═O)R^{a}, —C(═O)R^{a}, —C(═O)OR^{a}, —NR^{a}Rb, —NR^{a}(C═O)R^{b}, or halogen, where R^{a} and R^{b} represent independently of each other —H, —(C1-C10)alkyl, —(C1-C10)alkenyl, —(C1-C10)alkynyl, mono or polyfluorinated (C1-C10)alkyl, and -aryl, and R⁶ and R⁸ form together a (C3-C10)cycloalkyl or (C3-C10)heterocycloalkyl substituted by R^{c} and R^{d}, where R^{c} and R^{d} represent independently of each other —H, —(C1-C10)alkyl, —(C1-C10)alkenyl, —(C1-C10)alkynyl, mono or polyfluorinated (C1-C10)alkyl, -aryl, —OH, —O—(C1-C10)alkyl, —O—aryl, —O(C═O)—(C1-C10)alkyl, —O(C═O)-aryl, or halogen, or;
(iv) R⁵ and R⁷ form together a bond and R⁶ and R⁸ form together a (C3-C10)cycloalkyl or heterocycloalkyl substituted by R^{c} and R^{d} where R^{c} and R^{d} represent independently of each other —H, —(C1-C10)alkyl, —(C1-C10)alkenyl, —(C1-C10)alkynyl, mono or polyfluorinated (C1-C10)alkyl, -aryl, —OH, —O—(C1-C10)alkyl, —O—aryl, —O(C═O)—(C1-C10)alkyl, —O(C═O)-aryl, halogen, or;
(v) R⁵ and R⁷ form together a bond and R⁶ and R⁸ form together a (C6-C10)aryl or (C5-C10)heteroaryl substituted by R^{c} and R^{d}, where R^{c} and R^{d} represent independently of each other —H, —(C1-C10)alkyl, —(C1-C10)alkenyl, —(C1-C10)alkynyl, mono or polyfluorinated (C1-C10)alkyl, -aryl, —OH, —O—(C1-C10)alkyl, —O—aryl, —O(C═O)—(C1-C10)alkyl, —O(C═O)-aryl, or halogen, or;
(vi) when B¹ and B² form together —C═C—, R³ and R⁵ form together a bond and A³ is —C(R′R″)— or —N(R′)— AND R⁷ and R′ form together a bond, where R′ and R″ are independently of each other selected from —H, —(C1-C10)alkyl, -halogen, and —CF₃;
with the provisos that
1) when A¹, A² and A³ are —C(═O)— AND B¹ and B² are —O— AND R³, R⁵, R⁷ and R⁹ are —H, the compound of formula I excludes the compound of formula II 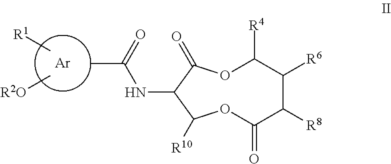
wherein
Ar is an aromatic ring selected from benzene, pyridine, indazole, benzotriazole, benzymidazole and pyrazolothiophene;
R¹ is selected from H, —C(1-10)alkyl, —O(C1-C10)alkyl, —Cl, —NO₂, —NH₂, —NHC(═O)H, —NH(C═O)—C(1-10)alkyl, —N[(C1-C10)alkyl]₂, —N[C(1-10)alkyl]C(═O)C(1-10)alkyl, and —NH(C═O)(CF₃(OMe))Ph;
R is selected from H, —C(1-10)alkyl, phenacyl, benzyl, benzoyl, and —(C═O)(CF₃(OMe))Ph;
R⁴ and R¹⁰ are independently of each other selected from —H and —(C1-C10)alkyl;
R⁶ and R⁸ are independently of each other selected from —H, —(C1-C10)alkyl , —OH, —O(C1-C10)alkyl, —OC(═O)(C1-C10)alkyl, —O—benzyl, —OC(═O)aryl, —OCO(CH₃)═CHCH₃, —CH₂C₆H₅, and —O(C═O)benzyl, wherein —(C1-C10)alkyl, —O(C1-C10)alkyl, —OC(═O)(C1-C10)alkyl, —O—benzyl, —OC(═O)aryl and —O(C═O)benzyl are optionally substituted with —OH, -halogen, —NO₂, or —CO₂H;
2) when A¹, A² and A³ are —C(═O)— AND B¹ and B² are —O—AND R³ and R⁹ are —H and R⁵ and R⁷ form together a bond and R⁶ and R⁸ form together an aryl ring, the compound of formula I excludes the compound of formula III 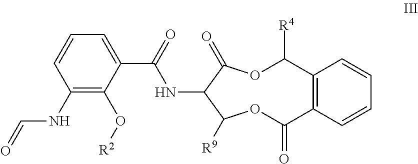
wherein
R², R⁴ and R⁹ are independently of each other selected from the group consisting of —H and —(C1-C10)alkyl;
3) when A¹ is —C(═O)— and A² is —CH₂— and A³ is —C(R′R″)— AND R⁹ and R¹⁰ are —H and B¹ and B² form together a —C═C— and R³ and R⁵ and R⁷ and R′ form together a bond, the compound of formula I excludes the compound of formula IV 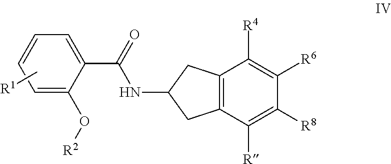
wherein
R″, R⁴, R⁶ and R⁸ are independently of each other selected from —H and —(SO₂)—R^{c}, where R^{c} is selected from -halogen, —(C1-C10)alkyl, -cycloalkyl, heterocycloalkyl, -aryl, -heteroaryl, —NH₂, —OH and a combination thereof;
R¹ is selected from H, —(C1-C10)alkyl, -aryl, —OH, —O—(C1-10)alkyl, —O— aryl, —NH₂, —NH(CHO), —NH(C═O)—(C1-10)alkyl, halogen, —NO₂, —C(═O)OH, —C(═O)O(C1-10)alkyl, —CF₃, and —NH(C═O)(CF₃(OMe))Ph;
R² is selected from H, —(C1-10)alkyl, aryl, benzyl, and —(C═O)(CF₃(OMe))Ph;
4) when A¹ is —C(═O)— and A³ is —N(R′)— AND R⁹ and R¹⁰ are —H and B¹ and B² form together a —CH—N— fragment and R³, R⁶ and R⁷ are —H, the compound of formula I excludes the compound of formula V 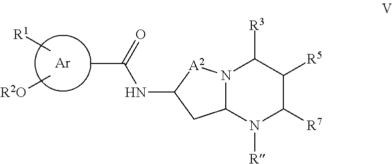
wherein
A² is selected from —CH₂— and —C(═O)—
Ar is selected from a (C5-C10)aromatic ring and a (C5-C10)heteroaromatic ring;
R¹ is selected from H, —(C1-C10)alkyl, -aryl, —OH, —O—(C1-10)alkyl, —O—aryl, —NH₂, —NH(CHO), —NH(C═O)—(C1-10)alkyl, halogen, —NO₂, —C(═O)OH, —C(═O)O(C1-10)alkyl, —CF₃, —NH(C═O)(CF₃(OMe))Ph;
R² is selected from H, —(C1-10)alkyl, aryl, benzyl, and —(C═O)(CF₃(OMe))Ph;
R³ is selected from —H, —(C1-10)alkyl, and —CF₃, or can form a bond with R⁵;
R⁵ and R⁷ are selected from independently of each other, —R^{a}, —OR^{a}, —O(C═O)R^{a}, —C(═O)R^{a}, —C(═O)OR^{a}, —NR^{a}R^{b}, —NR^{a}(C═O)R^{b}, and halogen, where R^{a} and R^{b} are independently of each other selected from —H, —(C1-C10)alkyl, —(C1-C10)alkenyl, —(C1-C10)alkynyl, mono or polyfluorinated (C1-C10)alkyl, —(C3-C10)cycloalkyl, —(C3-C10)heterocycloalkyl, -aryl, —(C1-C10)alkylaryl, —(C1-C10)alkylheteroaryl, —(C1-C10)alkyl-heterocycloalkyl and a combination thereof, or R⁵ and R⁷ form together a bond.
5) when A¹ is —(C═O)—; B¹ and B² form together —C═C—, R³ and R⁵ form together a bond; A³ is —N(R′)— where R⁷ and R′ form together a bond, then A² is not —CH₂—.

37. The compound of claim 36, wherein Ar is selected from (C5-C6)aromatic ring or (C5-C6)heteroaromatic ring where one or more of the carbon atoms in the ring system are replaced by heteroatoms selected from the group consisting of O, S, and N.

38. The compound of claim 37, wherein Ar is (C5-C6)aromatic ring.

39. The compound of claim 36, wherein A1 is —(C═O)—.

40. The compound of claim 36, wherein A² and A³ are —C(═O)—.

41. The compound of claim 36, wherein B¹ and B² are independently of each other selected from —O—, —S—, —NH—, —CH₂—, with the proviso that at least one of B¹ and B² is not —O—.

42. The compound of claim 36, wherein R⁶ and R⁸ form together a (C3-C10)cycloalkyl or (C3-C10)heterocycloalkyl substituted by R^{c} and R^{d}, where R^{c} and R^{d} are independently of each other selected from —H, —(C1-C10)alkyl, —(C1-C10)alkenyl, —(C1-C10)alkynyl, mono or polyfluorinated (C1-C10)alkyl, -aryl, —OH, —O—(C1-C10)alkyl, —O—aryl, —O(C═O)—(C1-C10)alkyl, —O(C═O)-aryl, and halogen.

43. The compound of claim 36, wherein R⁵, R⁸ and R¹⁰ are —H.

44. The compound of claim 36, wherein R⁵ and R⁷ form a bond together.

45. The compound of claim 44, wherein A² and A³ are —C(═O)— and R¹⁰ is H.

46. The compound of claim 44, wherein R⁶ and R⁸ form together a (C3-C10)cycloalkyl ring or a (C3-C10)heterocycloalkyl ring substituted with R^{c} and R^{d}, where R^{c} and R^{d} represent independently of each other —H, —(C1-C10)alkyl, —(C1-C10)alkenyl, —(C1-C10)alkynyl, mono or polyfluorinated (C1-C10)alkyl, -aryl, —OH, —O—(C1-C10)alkyl, —O—aryl, —O(C═O)—(C1-C10)alkyl, —O(C═O)-aryl, or halogen.

47. The compound of claim 44, wherein R⁶ and R⁸ form together a (C6-C10)aryl group or a (C5-C10)heteroaryl group substituted with R^{c} and R^{d}, where R^{c} and R^{d} represent, independently of each other —H, —(C1-C10)alkyl, —(C1-C10)alkenyl, —(C1-C10)alkynyl, mono or polyfluorinated (C1-C10)alkyl, -aryl, —OH, —O—(C1-C10)alkyl, —O-aryl, —O(C═O)—(C1-C10)alkyl, —O(C═O)-aryl, halogen.

48. The compound of claim 44, wherein R¹⁰ is —H; and A² and A³ are independently of each other selected from —(C═O)—, —(C═S)—, —(S═O)—, —(S(═O)₂)—, —(CH₂)—, —(C(CH₃)₂)—, —CH(CF₃)—, —CHF—, —(C(CH₂OCH₂))—, —(C(CH₂SCH₂))—, —N(R^{a})—, —(CR′R″)—, —CH₂—CH₂—, and —(C(CH₃)₂)—, where R′ and R″ are independently of each other selected from —H, —(C1-C10)alkyl, -halogen, and —CF₃, with the proviso that A² and A³ cannot be —C(═O)— at the same time.

49. The compound of claim 36, wherein R¹⁰ is —H; and B¹ and B² form together —CH—CH—, —C═C—, —CH—N—, or —N—N— forming respectively a bicyclic scaffold.

50. The compound of claim 49, wherein B¹ and B² form together —CH—CH— to provide a (3,4,0) bicycle.

51. The compound of claim 49, wherein B¹ and B² form together —C═C— providing a bicyclic scaffold; R³ and R⁵ form together a bond; and A³ is —C(R′R″)— or —N(R′)— and R⁷ with R′ form together a bond.

52. The compound of claim 36, wherein A² and A³ are independently of each other selected from —(C═O)—, —(C═S)—, —(S═O)—, —(S(═O)₂)—, —(CH₂)—, —(C(CH₃)₂)—, —CH(CF₃)—, —CHF—, —(C(CH₂OCH₂))—, —(C(CH₂SCH₂))—, —N(R′)—, —(CR′R″)—, —CH₂—CH₂—, and —(C(CH₃)₂)—, where R′ and R″ are selected from —H, —(C1-C10) alkyl, halogen, and —CF₃, with the proviso that A² and A³ cannot be —C(═O)— at the same time.

53. The compound of claim 52, wherein R⁵, R⁷ and R¹⁰ are —H.

54. The compound of claim 36, wherein B¹ and B² are independently of each other selected from —O—, —S—, —NH—, —CH₂—, with the proviso that at least one of B¹ and B² is not —O—.

55. The compound of claim 54, wherein R⁵, R⁷ and R¹⁰ are —H.

56. The compound of claim 36, wherein A² and A³ are —C(═O)—; B¹ and B² are —O—; R⁵, R⁷ and R¹⁰ are —H; with the proviso that at least one of the side chains R⁶ and R⁸ contains a —(C1-C10)alkyl-heterocycloalkyl group or a mono/polyfluorinated (C1-C10)alkyl.

57. The compound of claim 36, having formula IV

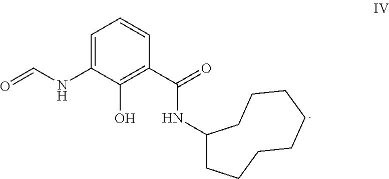 [SMILES: O=CNc1cccc(C(=O)NC2CCCCCCCC2)c1O ; "."]

58. A method for treating and/or preventing cancers presenting tumor-initiating cells in a patient comprising administering to said patient a therapeutically effective amount of a compound of formula I 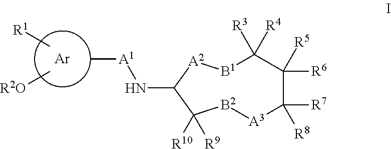
or a pharmaceutically acceptable salt or solvate thereof, wherein
Ar is selected from a (C5-C10)aromatic ring and a (C5-C10)heteroaromatic ring where one or more of the carbon atoms in the ring system are replaced by heteroatoms selected from the group consisting of O, S, and N;
R¹ is selected from H, —(C1-C10)alkyl, -aryl, —OH, —O—(C1-10)alkyl, —O—aryl, —NH₂, —NH(CHO), —NH(C═O)—(C1-10)alkyl, halogen, —NO₂, —C(═O)OH, —C(═O)O(C1-10)alkyl, —CF₃, and —NH(C═O)(CF₃(OMe))Ph;
R² is selected from H, —(C1-10)alkyl, aryl, benzyl, and —(C═O)(CF₃(OMe))Ph;
R³ is selected from —H, —(C1-10)alkyl, and —CF₃, or can form a bond with R⁵;
R⁴, R⁹ and R¹⁰ are independently of each other selected from —H, —(C1-10)alkyl, and —CF₃;
A¹, A² and A³ are independently of each other selected from —(C═O)—, —(C═S)—, —(S═O)—, —(S(═O)₂)—, —(CH₂)—, —(C(CH₃)₂)—, —CH(CF₃)—, —CHF—, —(C(CH₂OCH₂))—, —(C(CH₂SCH₂))—, —N(R^{a}), —(CR′R″)—, —CH₂—CH₂—, and —(C(CH₃)₂)—, where R^{a} is selected from —H, —(C1-C10)alkyl, —(C1-C10)alkenyl, —(C1-C10)alkynyl, mono or polyfluorinated (C1-C10)alkyl, —(C3-C10)cycloalkyl, —(C3-C10)heterocycloalkyl, -aryl, —(C1-C10)alkylaryl, —(C1-C10)alkylheteroaryl, and —(C1-C10)alkyl-heterocycloalkyl, and where R′ and R″ are independently of each other selected from —H, —(C1-C10)alkyl, -halogen, and —CF₃;
B¹ and B²:
(i) are independently of each other selected from —O—, —S—, —NH—, and —CH₂—, or
(ii) form together —CH—CH— producing a bicyclo(3,4,0)-nonane unit;
(iii) form together —C═C— producing a bicycle;
(iv) form together a —N—N— or —CH—N— fragment producing a diazabycycle or azabicycle;
R⁵, R⁶, R⁷ and R⁸ are selected from:
(i) independently of each other, —R^{a}, —OR^{a}, —O(C═O)R^{a}, —C(═O)R^{a}, —C(═O)OR^{a}, —NR^{a}R^{b}, —NR^{a}(C═O)R^{b}, and halogen, where R^{a} and R^{b} are independently of each other selected from —H, —(C1-C10)alkyl, —(C1-C10)alkenyl, —(C1-C10)alkynyl, mono or polyfluorinated (C1-C10)alkyl, -(C3-C10)cycloalkyl, —(C3-C10)heterocycloalkyl, -aryl, —(C1-C10)alkylaryl, —(C1-C10)alkylheteroaryl, —(C1-C10)alkyl-heterocycloalkyl and a combination thereof, or;
(ii) R⁵ and R⁷ form together a bond and R⁶ and R⁸ are independently of each other selected from —R^{a}, —OR^{a}, —O(C═O)R^{a}, —C(═O)R^{a}, —C(═O)OR^{a}, —NR^{a}R^{b}, —NR^{a}(C═O)R^{b}, and halogen, where R^{a} and R^{b} are independently of each other selected from —H, —(C1-C10)alkyl, —(C1-C10)alkenyl, —(C1-C10)alkynyl, mono or polyfluorinated (C1-C10)alkyl, —(C3-C10)cycloalkyl, —(C3-C10)heterocycloalkyl, -aryl, —(C1-C10)alkylaryl, —(C1-C10)alkylheteroaryl, —(C1-C10)alkyl-heterocycloalkyl and a combination thereof, or;
(iii) R⁵ and R⁷ represent independently of each other, —R^{a}, —OR^{a}, —O(C═O)R^{a}, —C(═O)R^{a}, —C(═O)OR^{a}, —NR^{a}R^{b}, —NR^{a}(C═O)R^{b}, or halogen, where R^{a} and R^{b} represent independently of each other —H, —(C1-C10)alkyl, —(C1-C10)alkenyl, —(C1-C10)alkynyl, mono or polyfluorinated (C1-C10)alkyl, or -aryl, and R⁶ and R⁸ form together a (C3-C10)cycloalkyl or (C3-C10)heterocycloalkyl substituted by R^{c} and R^{d} where R^{c} and R^{d} represent independently of each other —H, —(C1-C10)alkyl, —(C1-C10)alkenyl, —(C1-C10)alkynyl, mono or polyfluorinated (C1-C10)alkyl, -aryl, —OH, —O—(C1-C10)alkyl, —O—aryl, —O(C═O)—(C1-C10)alkyl, —O(C═O)-aryl, or halogen, or;
(iv) R⁵ and R⁷ form together a bond and R⁶ and R⁸ form together a (C3-C10)cycloalkyl or heterocycloalkyl substituted by R^{c} and R^{d} where R^{c} and R^{d} represent independently of each other —H, —(C1-C10)alkyl, —(C1-C10)alkenyl, —(C1-C10)alkynyl, mono or polyfluorinated (C1-C10)alkyl, -aryl, —OH, —O—(C1-C10)alkyl, —O—aryl, —O(C═O)—(C1-C10)alkyl, —O(C═O)-aryl, or halogen, or;
(v) R⁵ and R⁷ form together a bond and R⁶ and R⁸ form together a (C6-C10)aryl or a (C5-C10)heteroaryl substituted by R^{c} and R^{d} where R^{c} and R^{d} represent independently of each other —H, —(C1-C10)alkyl, —(C1-C10)alkenyl, —(C1-C10)alkynyl, mono or polyfluorinated (C1-C10)alkyl, -aryl, —OH, —O—(C1-C10)alkyl, —O—aryl, —O(C═O)—(C1-C10)alkyl, —O(C═O)-aryl, or halogen, or;
(vi) when B¹ and B² form together —C═C—, R³ and R⁵ form together a bond and A³ is —C(R′R″)— or —N(R′)— AND R⁷ and R′ form together a bond, where R′ and R″ are independently of each other selected from —H, —(C1-C10)alkyl, -halogen, and —CF₃;
with the provision that Antimycin A is excluded.

59. The method of claim 58, wherein said patient has undergone a prior removal of a tumor/cancer bulk.

60. The method of claim 58, wherein said cancers presenting tumor-initiating cells are selected from the group consisting of human gliomas, schwanommas, metastasis to the brain, meningiomas, ependymomas, and metastatic cancer.

61. The method of claim 60, wherein a metastatic cancer is selected from the group consisting of melanoma, breast cancer, colon cancer and lung cancer.

62. The method of claim 58, wherein Ar is selected from a (C5-C6)aromatic ring and a (C5-C6)heteroaromatic ring where one or more of the carbon atoms in the ring system are replaced by heteroatoms selected from the group consisting of O, S, and N.

63. The method of claim 62, wherein Ar is a (C5-C6)aromatic ring.

64. The method of claim 58, wherein said compound is of formula VI

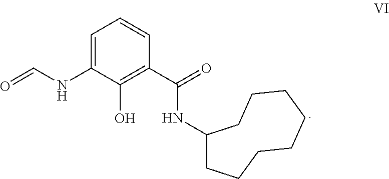 [SMILES: O=CNc1cccc(C(=O)NC2CCCCCCCC2)c1O ; "."]

65. A pharmaceutical composition comprising a therapeutically effective amount of the compound of claim 36 and a pharmaceutically acceptable carrier.

66. The pharmaceutical composition of claim 65, further comprising one or more additional active agents.
